(19) United States
(12) Patent Application Publication
(10) Pub. No.: US 2007/0292476 A1
(43) Pub. Date: Dec. 20, 2007

(54) Delivery of ophthalmologic agents to the exterior or interior of the eye

(75) Inventors: Geoffrey C. Landis, Carlsbad, CA (US); William G. Turnell, Del Mar, CA (US); Yuan Yumin, San Diego, CA (US)

Correspondence Address: DLA PIPER US LLP, 4365 EXECUTIVE DRIVE, SUITE 1100, SAN DIEGO, CA, 92121-2133, US

(73) Assignee: MediVas, LLC, San Diego, CA (US)

(21) Appl. No.: 11/799,725

(22) Filed: May 1, 2007

Related U.S. Application Data
(60) Provisional application No. 60/797,339, filed on May 2, 2006.

(30) Foreign Application Priority Data
Sep. 21, 2006 (US) PCT/US06/36935

(51) Int. Cl. A61F 2/00; A61K 31/047; A61P 27/02

(52) U.S. Cl. 424/428; 514/738

(57) ABSTRACT

The present invention provides intraocular polymer delivery compositions based on biodegradable polyester amide (PEA), polyester urethane (PEUR), and polyester urea (PEU) polymers, which contain amino acids. The compositions can be formulated as an implantable solid or as a liquid dispersion of polymer particles for sustained delivery of ophthalmologic agents dispersed therein or incorporated into the backbone of the polymers. Methods of delivering an ophthalmologic agent to the exterior or interior of the eye by implanting the composition in the eye of a subject are also included.

### RELATED APPLICATIONS

This application claims priority under §35 U.S.C. 119(e) of Ser. Nos. 60/684,670, filed May 25, 2005; 60/737,401, filed Nov. 14, 2005; 60/687,570, filed Jun. 3, 2005; 60/759,179, filed Jan. 13, 2006; 60/719,950, filed Sep. 22, 2005; 60/719,809, filed Sep. 22, 2005, and 60/797,339, filed May 2, 2006, under §35 U.S.C. 120 of Ser. No. 11/344,689, filed Jan. 31, 2006, and under §35 U.S.C. 365 of PCT/US2006/036935, filed Sep. 21, 2006; and this application is a continuation in part application under 35 U.S.C. §120 of U.S. Ser. No. 10/362,848, filed Oct. 14, 2003, which is a continuation of U.S. Pat. No. 6,503,538 B1, each of which is incorporated herein by reference in its entirety.

### FIELD OF THE INVENTION

The invention relates generally to drug-eluting polymer compositions and in particular to biodegradable, biocompatible polymer delivery compositions for ocular delivery of ophthalmologic agents in a controlled time release fashion.

### BACKGROUND INFORMATION

Many implantable drug delivery devices have been developed over the past several years. Such drug delivery devices may be formulated from synthetic or natural, biodegradable or non-biodegradable, polymers. Biodegradable polymers are preferred since these materials gradually degrade in vivo over time, e.g., by enzymatic or non-enzymatic hydrolysis, when placed in an aqueous, physiological environment. Thus, the use of biodegradable polymers in drug delivery devices is preferred since their use avoids the necessary removal of the drug delivery device at the end of the drug release period.

The drug is generally incorporated into the polymeric composition and formed into the desired shape outside the body. This solid implant is then typically inserted into the body of a human, animal, bird, and the like through an incision. Alternatively, small discrete particles composed of these polymers can be injected into the body by a syringe.

Certain of these polymers also can be injected via syringe as a liquid polymeric composition. These compositions are administered to the body in a liquid state or, alternatively, as a solution. Once in the body, the composition coagulates into a solid. One type of polymeric composition includes a nonreactive thermoplastic polymer or copolymer dissolved in a body fluid-dispersible solvent. This polymeric solution is placed into the body where the polymer congeals or precipitatively solidifies upon dissipation or diffusion of the solvent into the surrounding body tissues.

In particular, nonbiodegradable polymer implants, most commonly various types of methylmethacrylate, have been used for local delivery of antibiotics, such as Tobramycin, gentamicin, and vancomycin. A biodegradable antibiotic implant made of polylactic acid and poly(DL-lactide): co-glycolide combined with vancomycin has been developed and evaluated in a localized osteomyelitic rabbit model (Cahoun J H et al. Clinical Orthopaedics & Related Research. Current Trends in the Management of Disorders of the Joints. (1997) 341: 206-214).

More recently the GIADEL® wafer (Guilford Pharmaceutical Corp, Baltimore, Md.), which was FDA-approved for implant in post surgical treatment of certain kinds of brain tumors, is used to deliver an oncolytic agent, carmustine, from a wafer of a biodegradable polyanhydride copolymer. Hydrolytic degradation products of Gliadel® wafer (in addition to the anticancer agent) are ultimately the starting di-acids: sebacic acid and 1,3-bis(4-carboxyphenoxy) propane (CPP). Clinical investigations of Gliadel implants in rabbit brains have shown limited toxicity, initial activity and fast excretion of decomposition products - the free acids (A. J. Domb et al. Biomaterials. (1995) 16: 1069-1072).

Local drug delivery from implants provides the advantage of high tissue concentrations with relatively low serum levels, thereby avoiding some of the toxicity associated with systemic delivery. Bioactive agent-impregnated polymer implants are particularly attractive because they not only deliver high tissue levels of antibiotic or oncolytic agent, but also help fill the dead space that occurs after certain surgeries. However, drug release characteristics of such implanted drug delivery devices may be suboptimal. Many times, the release of pharmacologically active agents from an implanted drug delivery device is irregular. There is an initial burst period when the drug is released primarily from the surface of the device, followed by a second period during which little or no drug is released, and a third period during which most of the remainder of the drug is released at a substantially lower rate than in the initial burst.

More recently CPP was disclosed as a monomer useful in preparation of bioabsorbable stents for vascular applications by “Advanced Cardiovascular Systems, Inc”, in patent WO 03/080147 A1, 2003 and polymer particles in co-pending provisional application Ser. No. 60/684,670, filed May 25, 2005.

Another aromatic biodegradable di-acid monomer based on trans-4-hydroxycinnamic acid has been recently described. The monomer with general name 4,4′-(alkanedioyidioxy) dicinnamic acid inherently contains two hydrolytically labile ester groups, and is expected to undergo specific (enzymatic) and nonspecific (chemical) hydrolysis (M Nagata, Y. Sato. Polymer. (2004) 45: 87-93). The biodegradable polymers containing unsaturated groups have potential for various applications. For example, unsaturated groups can be converted into other functional groups such as epoxy or alcohol—useful for further modifications. Their crosslinking could enhance thermal stability and mechanical properties of polymer. Cinnamate is known to undergo reversible [2+2] cyclo-addition upon UV irradiation at wavelengths over 290 nm, without presence of photoinitiator, a property which makes the polymer self-photo-crosslinkable (Y. Nakayama, T. Matsuda. J. Polym. Sci. Part A: Polym. Chem. (1992) 30: 2451-2457). In addition, the cinnamoyl esters are metabolized in the body and have been proven to be non-toxic (Citations in paper of M Nagata, Y. Sato. Polymer. (2004) 45: 87-93).

Recent research has also shown that hydrogel-type materials can be used to shepherd various medications through the stomach and into the more alkaline intestine. Hydrogels are cross-linked, hydrophilic, three-dimensional polymer networks that are highly permeable to various drug compounds, can withstand acidic environments, and can be tailored to “swell” and thereby release entrapped molecules through their weblike surfaces. Depending on the chemical composition of the gel, different internal and external stimuli (e.g., changes in pH, application of a magnetic or electric field, variations in temperature, and ultrasound irradiation) may be used to trigger the swelling effect. Once triggered, however, the rate of entrapped drug release is determined solely by the cross-linking ratio of the polymer network.

Delivery of drugs intraocularly is a particular problem. The eye is divided into two chambers; the anterior segment which is the front of the eye, and the posterior segment which is the back of the eye. Diseases of the anterior segment are easier to treat with formulations such as eye drops because they can be applied topically. For example, glaucoma can be treated from the front of the eye. Diseases of the retina, such as diabetic retinopathy and macular degeneration, are located in the posterior segment and are difficult to treat because drugs applied topically, such as eye drops, typically do not penetrate to the back of the eye. Drugs for these disease states have customarily been delivered by injection directly into the back of the eye.

Researchers have sought to overcome these difficulties. The topical administration of a cationic emulsion onto the eye has been shown to increase the residence time of a lipophilic drug on the cornea, with a lower contact angle and an increased spreading coefficient in comparison with conventional eye drops and anionic emulsions. In the case of the posterior segment of the eye, certain cationic emulsions for non-invasive topical administration have been developed that allow a lipophilic drug to migrate to the retina via the trans-scleral route from the cornea and conjunctiva, which act as a reservoir.

Chemists, biochemists, and chemical engineers are all looking beyond traditional polymeric and other formulations to find innovative drug transport systems. Thus, there is still a need in the art for new and better polymer delivery compositions for controlled delivery of a variety of different types of bioactive agents to target specific body sites, such as the exterior and interior tissues of the eye. In particular, there is a need in the art for new and better polymer delivery compositions for continuous delivery of an ophthalmologic agent to the anterior or posterior segment of the eye over a sustained period of time, for example in treatment of chronic diseases of the front and back of the eye.

### SUMMARY OF THE INVENTION

The present invention is based on the premise that polymers containing at least one amino acid and a moiety that is not an amino acid per repeat unit, such as polyester amide (PEA) polyester urethane (PEUR) and polyester urea (PEU) polymers, can be used to formulate biodegradable polymer delivery compositions for time release of ophthalmologic agents in a consistent and reliable manner into the exterior or interior of the eye by biodegradation of the polymer.

The present invention is also based on the premise that PEAs, PEURs and PEUs can be formulated as polymer delivery compositions that incorporate a therapeutic agent (e.g., a residue of an ophthalmologic diol) into the backbone of the polymer for time release into the exterior or interior of the eye in a consistent and reliable manner by biodegradation of the polymer. The invention intraocular polymer delivery composition may optionally also deliver into the exterior or interior of the eye another type of bioactive agent that is dispersed in the polymer.

In one embodiment, the invention provides an intraocular polymer delivery composition comprising at least one ophthalmologic agent dispersed in at least one biodegradable polymer, wherein the composition is implantable in the exterior or interior of the eye and the polymer comprises at least one of a PEA having a chemical formula described by structural formula (I), 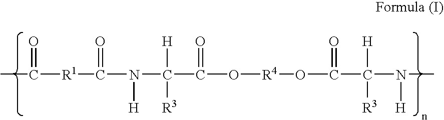
wherein n ranges from about 5 to about 150; R¹ is independently selected from residues of α,ω-alkylene dicarboxylates of formula (III) below, or in combination with (C₂- C₂₀) alkylene, (C₂-C₂₀) alkenylene, α,ω-bis(4-carboxyphenoxy)-(C₁-C₈) alkane, 3,3′-(alkanedioyldioxy) dicinnamic acid or 4,4′-(alkanedioyldioxy) dicinnamic acid, or saturated or unsaturated residues of therapeutic di-acids; and wherein R⁵ and R⁶ in formula (III) are independently selected from (C₂-C₁₂) alkylene or (C₂-C₁₂) alkenylene; the R³s in individual n units are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), saturated or unsaturated therapeutic diol residues, and combinations thereof; 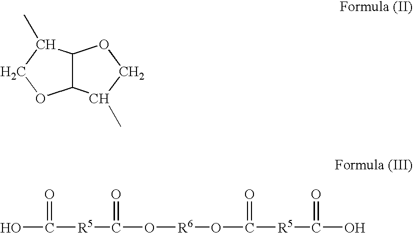
or a PEA polymer having a chemical formula described by structural formula (IV): 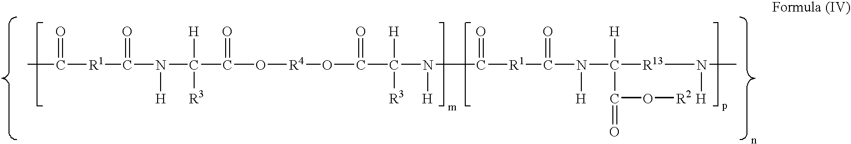
   wherein n ranges from about 5 to about 150, m ranges about 0.1 to 0.9; p ranges from about 0.9 to 0.1; R¹ is independently selected from residues of α,ω-alkylene dicarboxylates of structural formula (III), or in combination with (C₂- C₂₀) alkylene and (C₂-C₂₀) alkenylene, α,ω-bis(4-carboxyphenoxy)-(C₁-C₈) alkane, 3,3′-(alkanedioyidioxy) dicinnamic acid, 4,4′-(alkanedioyidioxy)dicinnamic acid, saturated or unsaturated residues of therapeutic di-acids and combinations thereof; and wherein R⁵ and R⁶ in Formula (III) are independently selected from (C₂- C₁₂) alkylene or (C₂-C₁₂) alkenylene; each R² is independently hydrogen, (C₁-C₁₂) alkyl, (C₆-C₁₀) aryl or a protecting group; the R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated or unsaturated therapeutic diols, and combinations thereof: and R¹³ is independently (C₁-C₂₀) alkyl or (C₂-C₂₀) alkenyl.

In still another embodiment, the invention provides methods for delivering an ophthalmologic agent to the exterior or interior of the eye of a subject by administering to the subject ocularly an invention intraocular polymer delivery composition comprising one or more polymers of structural formulas I and IV-VIII and at least one ophthalmologic agent dispersed in said one or more polymers, which composition biodegrades by enzymatic action to release the ophthalmologic agent(s) to the exterior or interior of the eye at a controlled rate.

### DETAILED DESCRIPTION OF THE INVENTION

The invention is based on the discovery that biodegradable polymers can be used to create a polymer delivery composition for ocular delivery of ophthalmologic agents dispersed within. The ocular polymer delivery compositions can be fashioned as dispersions of particles, as implantable solids, or as films for extra- or intraocular delivery. The invention ocular polymer delivery compositions biodegrade by enzymatic hydrolytic actions so as to release the ophthalmologic agent at a controlled rate. The invention particle compositions are stable, and can be lyophilized for transportation and storage and be redispersed for administration. Due to structural properties of the polymer used, the invention intraocular polymer delivery compositions provide for high loading of ophthalmologic agents.

Accordingly, in one embodiment, the invention provides an intraocular polymer delivery composition comprising at least one ophthalmologic agent dispersed in at least one biodegradable polymer, wherein the polymer is a PEA having a chemical formula described by structural formula (I), 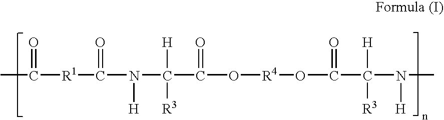
wherein n ranges from about 5 to about 150; R¹ is independently selected from residues of α,ω-bis (4-carboxyphenoxy) (C₁-C₈) alkane, 3,3′-(alkanedioyidioxy) dicinnamic acid or 4,4′-(alkanedioyldioxy) dicinnamic acid, residues of α,ω-alkylene dicarboxylates of formula (III), (C₂-C₂₀) alkylene, (C₂-C₂₀) alkenylene or a saturated or unsaturated residues of therapeutic di-acids and combinations thereof; and wherein R⁵ and R⁶ in Formula (III) are independently selected from (C₂- C₁₂) alkylene or (C₂-C₁₂) alkenylene; the R³s in individual n monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl and —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), and combinations thereof, (C₂-C₂₀) alkylene, (C₂-C₂₀) alkenylene, and saturated or unsaturated therapeutic di-acid residues; 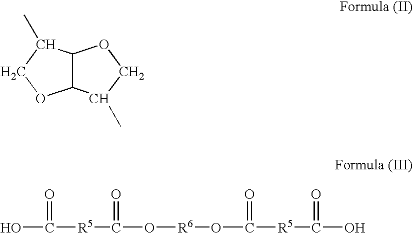
or a PEA polymer having a chemical formula described by structural formula (IV), 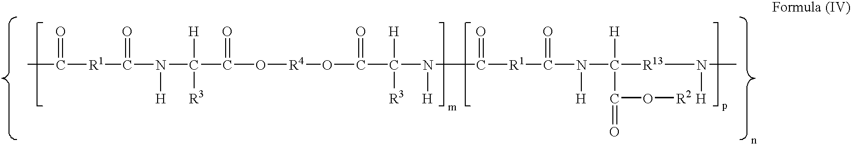
   wherein n ranges from about 5 to about 150, m ranges about 0.1 to 0.9: p ranges from about 0.9 to 0.1; wherein R¹ is independently selected from residues of α,ω-bis (4-carboxyphenoxy) (C₁-C₈)alkane, 3,3′-(alkanedioyldioxy)dicinnamic acid or 4,4′-(alkanedioyldioxy)dicinnamic acid, residues of α,ω-alkylene dicarboxylates of formula (III) above, (C₂-C₂₀) alkylene, (C₂-C₂₀) alkenylene or a saturated or unsaturated residues of therapeutic di-acids and combinations thereof; wherein R⁵ and R⁶ in Formula (III) are independently selected from (C₂-C₁₂) alkylene or (C₂-C₁₂) alkenylene; each R² is independently hydrogen, (C₁-C₁₂) alkyl or (C₆-C₁₀) aryl or a protecting group; the R³s in individual m monomers are independently selected from the group consisting of hydrogen, (C₁-C₆) alkyl, (C₂-C₆) alkenyl, (C₂-C₆) alkynyl, (C₆-C₁o) aryl (C₁-C₆) alkyl, and —(CH₂)₂S(CH₃); and R⁴ is independently selected from the group consisting of (C₂-C₂₀)alkylene, (C₂-C₂₀) alkenylene, (C₂-C₈) alkyloxy (C₂-C₂₀) alkylene, bicyclic-fragments of 1,4:3,6-dianhydrohexitols of structural formula (II), and combinations thereof, and residues of saturated or unsaturated therapeutic diols; and R¹³ is independently (C₁-C₂₀) alkyl or (C₂-C₂₀) alkenyl, for example, (C₃-C₆) alkyl or (C₃-C₆) alkenyl.

For example, an effective amount of the residue of at least one therapeutic diol or di-acid can be contained in the polymer backbone. For example, the therapeutic diol can be an ophthalmologic agent, as disclosed herein. Alternatively, in the PEA polymer, at least one R¹ is a residue of α,ω-bis (4-carboxyphenoxy) (C₁-C₈) alkane or 4,4′-(alkanedioyldioxy) dicinnamic acid and R⁴ is a bicyclic-fragment of a 1,4:3,6-dianhydrohexitol of general formula (II), or a residue of a saturated or unsaturated therapeutic diol. In another alternative, R¹ in the PEA polymer is either a residue of α,ω-bis (4-carboxyphenoxy) (C₁-C₈) alkane, or 4,4′-(alkanedioyl dioxy) dicinnamic acid, a residue of a therapeutic diacid, and mixtures thereof. In yet another alternative, in the PEA polymer R¹ is a residue α,ω-bis (4-carboxyphenoxy) (C₁-C₈) alkane, such as 1,3-bis(4-carboxyphenoxy)propane (CPP), or 4,4′-(adipoyldioxy) dicinnamic acid and R⁴ is a bicyclic-fragment of a 1,4:3,6-dianhydrohexitol of general formula (II), such as 1,4:3,6-dianhydrosorbitol (DAS).

Alternatively, the invention intraocular polymer delivery composition can comprise at least one ophthalmologic agent dispersed in a biodegradable polymer, wherein the polymer comprises a PEUR polymer having a chemical formula described by structural formula (V), 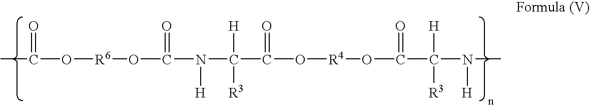
and wherein n ranges from about 5 to about 150; wherein the R³s within an individual n monomer are independently selected from the group consisting of hydrogen, (C₁-C₆) alkyl, (C₂-C₆) alkenyl, (C₂-C₆) alkynyl, (C₆-C₁₀) aryl(C₁-C₆) alkyl and —(CH₂)₂S(CH₃); R⁴ and R⁶ are independently selected from (C₂-C₂₀) alkylene, (C₂-C₂₀) alkenylene or (C₂-C₈) alkyloxy (C₂-C₂₀) alkylene, bicyclic-fragments of 1,4:3,6-dianhydrohexitols of general formula (II), a residue of a saturated or unsaturated therapeutic diol, and mixtures thereof. or a PEUR polymer having a chemical structure described by general structural formula (VI), 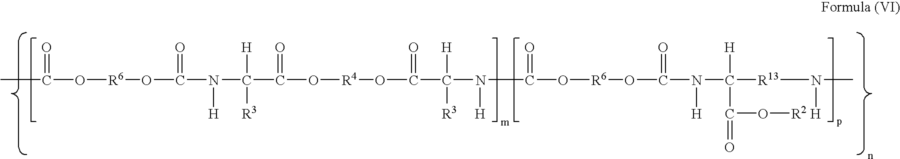
wherein n ranges from about 5 to about 150, m ranges about 0.1 to about 0.9: p ranges from about 0.9 to about 0.1; R² is independently selected from hydrogen, (C₆-C₁₀)aryl (C₁-C₆) alkyl, or a protecting group; the R³s within an individual m monomer are independently selected from the group consisting of hydrogen, (C₁-C₆) alkyl, (C₂-C₆) alkenyl, (C₂-C₆) alkynyl, (C₆-C₁₀) aryl(C₁-C₆) alkyl, and —(CH₂)₂S(CH₃); R⁴ and R⁶ are independently selected from (C₂-C₂₀) alkylene, (C₂-C₂₀) alkenylene or (C₂-C₈) alkyloxy (C₂-C₂₀) alkylene, bicyclic-fragments of 1,4:3,6-dianhydrohexitols of structural formula (II), a residue of a saturated or unsaturated therapeutic diol, and mixtures thereof; and R¹³ is independently (C₁-C₂₀) alkyl or (C₂-C₂₀) alkenyl, for example, (C₃-C₆) alkyl or (C₃-C₆) alkenyl.

For example, an effective amount of the residue of at least one therapeutic diol, including, but not limited to, an ophthalmologic diol, as disclosed herein, can be contained in the polymer backbone. In one alternative in the PEUR polymer, at least one of R⁴ or R⁶ is a bicyclic fragment of 1,4:3,6-dianhydrohexitol, such as 1,4:3,6-dianhydrosorbitol (DAS).

In still another embodiment the invention intraocular polymer delivery composition can comprise at least one ophthalmologic agent dispersed in a biodegradable polymer, wherein the polymer comprises at least one biodegradable PEU polymer having a chemical formula described by structural formula (VII), 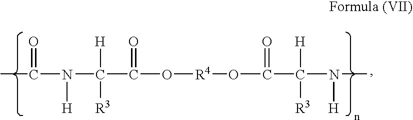
wherein n is about 10 to about 150; the R³s within an individual n monomer are independently selected from hydrogen, (C₁-C₆) alkyl, (C₂-C₆) alkenyl, (C₂-C₆) alkynyl, (C₆-C₁₀) aryl (C₁-C₆)alkyl, —(CH₂)₃, and —(CH₂)₂S(CH₃); R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II);
or a PEU having a chemical formula described by structural formula (VIII), 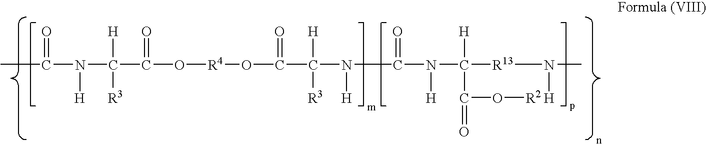
   wherein m is about 0.1 to about 1.0; p is about 0.9 to about 0.1; n is about 10 to about 150; each R² is independently hydrogen, (C₁-C₁₂) alkyl or (C₆-C₁₀) aryl; and the R³s within an individual m monomer are independently selected from hydrogen, (C₁-C₆) alkyl, (C₂-C₆) alkenyl, (C₂-C₆) alkynyl, (C₆-C₁₀) aryl (C₁-C₆)alkyl, and —(CH₂)₂S(CH₃); R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II), or a mixture thereof; and R¹³ is independently (C₁-C₂₀) alkyl or (C₂-C₂₀) alkenyl, for example, (C₃-C₆) alkyl or (C₃-C₆) alkenyl.

For example, an effective amount of the residue of at least one therapeutic diol, including but not limited to an ophthalmologic diol, as disclosed herein, can be contained in the polymer backbone. In one alternative in the PEU polymer, at least one R⁴ is a residue of a saturated or unsaturated therapeutic diol, or a bicyclic fragment of a 1,4:3,6-dianhydrohexitol, such as DAS. In yet another alternative in the PEU polymer, at least one R⁴ is a bicyclic fragment of a 1,4:3,6-dianhydrohexitol, such as DAS.

These PEU polymers can be fabricated as high molecular weight polymers useful for making polymer particles suitable for delivery into the interior or exterior of the eye of humans and other mammals of a variety of ophthalmologic agents. These PEUs incorporate hydrolytically cleavable ester groups and non-toxic, naturally occurring monomers that contain a-amino acids in the polymer chains. The ultimate biodegradation products of PEUs will be amino acids, diols, and CO₂. In contrast to the PEAs and PEURs, the invention PEUs are crystalline or semi-crystalline and possess advantageous mechanical, chemical and biodegradation properties that allow formulation of completely synthetic, and hence easy to produce, crystalline and semi-crystalline polymer particles, for example nanoparticles.

For example, the PEU polymers used in the invention intraocular polymer particle delivery compositions have high mechanical strength, and surface erosion of the PEU polymers can be catalyzed by enzymes present in physiological conditions, such as hydrolases.

As used herein, the terms “amino acid” and “α-amino acid” mean a chemical compound containing an amino group, a carboxyl group and a pendent R group, such as the R³ groups defined herein. As used herein, the term “biological α-amino acid” means the amino acid(s) used in synthesis are selected from phenylalanine, leucine, glycine, alanine, valine, isoleucine, methionine, or a mixture thereof. As used herein, the term “adirectional amino acid” means a chemical moiety within the polymer chain obtained from an α-amino acid, such that the R group (for example R¹³ in formulas VI and VII) is inserted within the polymer backbone.

As used herein, a “therapeutic diol” means any diol molecule, whether synthetically produced, or naturally occurring (e.g., endogenously), that affects a biological process in a mammalian individual, such as a human, in a therapeutic or palliative manner when administered to the mammal.

The PEA, PEUR and PEU polymers used in the invention compositions are poly-condensates. The ratios “m” and “p” in Formulas (IV, VI and VIII) are defined as irrational numbers in the description of these poly-condensate polymers. Moreover, as “m” and “p” will each take up a range within any poly-condensate, such a range cannot be defined by a pair of integers. Each polymer chain is a string of monomer residues linked together by the rule that all bis-amino acid diol (i) and adirectional amino acid (e.g. lysine) (ii) monomer residues are linked either to themselves or to each other by a diacid monomer residue (iii) for PEA, by a diol residue (iii) for PEUR or carbonyl for PEU (iii). Thus, only linear combinations of i-iii-i; i-iii-ii (or ii-iii-i) and ii-iii-ii are formed. In turn, each of these combinations is linked either to themselves or to each other by a diacid monomer residue (iii) for PEA or a diol residue (iii) for PEUR or carbonyl for PEU (iii). Each polymer chain is therefore a statistical, but non-random, string of monomer residues. Each individual polymer chain is composed of integer numbers of monomers, i, ii and iii. However, in general for polymer chains of any practical average molecular weight (i.e., sufficient mean length), the ratios of monomer residues “m” and “p” in formulas (IV, VI and VIII) will not be whole numbers (rational integers). Furthermore, for the condensate of all poly-dispersed polymer chains the numbers of monomers i, ii and iii averaged over all of the chains (i.e. normalized to the average chain length) will not be integers. It follows that the ratios can only take irrational values (i.e., any real number that is not a rational number). Irrational numbers, as the term is used herein, are derived from ratios that are not of the form n/j, where n and j are integers.

As used herein, the term “residue of a therapeutic diol” means a portion of a therapeutic diol, as described herein, which portion excludes the two hydroxyl groups of the diol. As used herein, the term “residue of a therapeutic di-acid” means a portion of a therapeutic di-acid, as described herein, which portion excludes the two carboxyl groups of the di-acid. The corresponding therapeutic diol or di-acid containing the “residue” thereof is used in synthesis of the polymer compositions. The ophthalmologic agents disclosed herein are a subset of the therapeutic diols disclosed herein. The residue of the therapeutic di-acid or diol is reconstituted in vivo (or under similar conditions of pH, aqueous media, and the like) to the corresponding di-acid or diol upon release from the backbone of the polymer by biodegradation in a controlled manner that depends upon the properties of the PEA, PEUR or PEU polymer(s) selected to fabricate the composition, which properties are as known in the art and as described herein.

As used herein the term “bioactive agent” means a bioactive agent as disclosed herein that is not incorporated into the polymer backbone. One or more such bioactive agents optionally may be dispersed in the invention intraocular polymer delivery compositions. As used herein, the term “dispersed” means that the bioactive agent is dispersed, mixed, dissolved, homogenized, and/or covalently bound to (“dispersed”) in a polymer, for example attached to a functional group in the biodegradable polymer of the composition or to the surface of a polymer particle, but not incorporated into the backbone of a PEA, PEUR, or PEU polymer. To distinguish backbone-incorporated therapeutic diols and di-acids from those that are not incorporated into the polymer backbone, (as a residue thereof), such dispersed therapeutic or palliative agents are referred to herein as “bioactive agent(s)” and may be contained within polymer conjugates or otherwise dispersed in the polymer particle composition, as described below. Such bioactive agents may include, without limitation, small molecule drugs, peptides, proteins, DNA, cDNA, RNA, sugars, lipids and whole cells. In one embodiment, the invention intraocular polymer delivery composition administers the ophthalmologic agent, with or without an optional bioactive agent dispersed therein in polymer particles having a variety of sizes and structures suitable to meet differing therapeutic goals and routes of administration.

The term, “biodegradable, biocompatible” as used herein to describe the PEA, PEUR and PEU polymers, including mixtures and blends thereof, used in fabrication of invention intraocular polymer delivery compositions means the polymer is capable of being broken down into innocuous products in the normal functioning of the body. This is particularly true when the amino acids used in fabrication of the polymers are biological L-α-amino acids. A “biodegradable polymer” as the term is used herein also means the polymer is degraded by water and/or by enzymes found in tissues of mammalian patients, such as humans. The invention intraocular polymer delivery compositions are also suitable as implants for use in veterinary treatment of a variety of mammalian patients, such as pets (for example, cats, dogs, rabbits, ferrets), farm animals (for example, swine, horses, mules, dairy and meat cattle) and race horses when used as described herein.

The term “controlled” as used herein to described the release of bioactive agent(s) from invention intraocular polymer delivery compositions means the polymer implant degrades over a desired period of time to provide a smooth and regular (i.e. “controlled”) time release profile (e.g., avoiding an initial irregular spike in drug release and providing instead a substantially smooth rate of change of release throughout biodegradation of the invention composition).

The polymers in the invention intraocular polymer delivery compositions include hydrolyzable ester and enzymatically cleavable amide linkages that provide biodegradability, and are typically chain terminated, predominantly with amino groups. Optionally, the amino termini of the polymers can be acetylated or otherwise capped by conjugation to any other acid-containing, biocompatible molecule, to include without restriction organic acids, bioinactive biologics, and bioactive agents as described herein. In one embodiment, the entire polymer composition, and any particles made thereof, is substantially biodegradable.

In one alternative, at least one of the α-amino acids used in fabrication of the polymers used in the invention compositions and methods is a biological α-amino acid. For example, when the R³s are CH₂Ph, the biological a-amino acid used in synthesis is L-phenylalanine. In alternatives wherein the R³s are CH₂—CH(CH₃)₂, the polymer contains the biological α-amino acid, L-leucine. By varying the R³s within monomers as described herein, other biological a-amino acids can also be used, e.g., glycine (when the R³s are H), alanine (when the R³s are CH₃), valine (when the R³s are CH(CH₃)₂), isoleucine (when the R³s are CH(CH₃)—CH₂—CH₃), phenylalanine (when the R³s are CH₂—C₆H₅), or methionine (when the R³s are —(CH₂)₂SCH₃), and mixtures thereof. In yet another alternative embodiment, all of the various α-amino acids contained in the polymers used in making the invention polymer particle or solid intraocular delivery compositions are biological a-amino acids, as described herein.

The term, “biodegradable” as used herein to describe the polymers used in the invention intraocular polymer delivery composition means the polymer is capable of being broken down into innocuous and bioactive products in the normal functioning of the body. In one embodiment, the entire polymer particle delivery composition is biodegradable. The biodegradable polymers described herein have hydrolyzable ester and enzymatically cleavable amide linkages that provide the biodegradability, and are typically chain terminated predominantly with amino groups. Optionally, these amino termini can be acetylated or otherwise capped by conjugation to any other acid-containing, biocompatible molecule, to include without restriction organic acids, bioinactive biologics and bioactive agents.

In one embodiment the invention intraocular polymer delivery compositions are fabricated as particles, which can be formulated to provide a variety of properties. For example, the polymer particles can be sized to agglomerate intraocularly, forming a time-release polymer depot for local delivery of dispersed ophthalmologic agents to surrounding tissue/cells when injected or surgically implanted therein. For example, polymer particles of sizes capable of passing through pharmaceutical syringe needles ranging in size from about 19 to about 27 gauge, for example those having an average diameter in the range from about 1 μm to about 200 μm, can be injected intraocularly, and will agglomerate to form particles of increased size that form the depot to dispense the ophthalmologic agent(s) locally.

The biodegradable polymers used in the invention intraocular polymer delivery composition can be designed to tailor the rate of biodegradation of the polymer to result in continuous delivery of the ophthalmologic agent, with or without an additional bioactive agent, over a selected period of time. For instance, typically, a polymer depot, as described herein, will biodegrade over a time selected from about twenty-four hours, about seven days, about thirty days, or about ninety days, or longer, for example up to three years. Longer time spans are particularly suitable for providing a delivery composition that eliminates the need to repeatedly inject the composition to obtain a suitable therapeutic or palliative response.

The invention utilizes biodegradable polymer particle- or solid-mediated delivery techniques to deliver ophthalmologic agents into the interior or exterior of the eye. For example the invention compositions can be placed subconjunctivally (under the thin membrane that covers the top of the front of the eye except for the cornea) but on top of the sclera (white part of the eye) to provide a sustained delivery. Alternatively, the invention composition can be placed surgically, as described herein, to delivery the ophthalmologic agent to the back of the eye. Alternatively still, the invention composition can be placed underneath the Tenon's capsule on top of the sclera to allow diffusion of the ophthalmologic agent through the sciera from the back of the eye to the retina. This mode of emplacement is called “subtenon” delivery. Thus, the invention compositions can be used to deliver ophthalmologic agents in treatment of a wide variety of ophthalmologic diseases and disease symptoms.

As used herein, the terms “intraocular”, “intraocularly” and “into the interior of the eye” mean subconjunctival, transcleral, or subtenon delivery of an active agent, but not topical delivery.

Although certain of the individual components of the polymer particle and solid delivery compositions and methods described herein were known, it was unexpected and surprising that such combinations would enhance the efficiency of time release delivery of the ophthalmologic agents beyond levels achieved when the components were used separately.

The PEA, PEUR and PEU polymers used in practice of the invention bear functionalities that allow facile covalent attachment to the polymer of the ophthalmologic agent and, optionally, other bioactive agent(s) or covering molecule(s). For example, a polymer bearing carboxyl groups can readily react with an amino moiety, thereby covalently bonding a peptide to the polymer via the resulting amide group. As will be described herein, the biodegradable polymer and the bioactive agent may contain numerous complementary functional groups that can be used to covalently attach an ophthalmologic or other bioactive agent to the biodegradable polymer. Alternatively, such polymers used in the invention compositions, such as the polymer particles, and methods are ready for reaction with other chemicals having a hydrophilic structure to increase water solubility and with covering molecules, without the necessity of prior modification.

In addition, the polymers disclosed herein (e.g., those having structural formulas (I and IV-VIII), upon enzymatic degradation, provide amino acids while the other breakdown products can be metabolized in the way that fatty acids and sugars are metabolized. Uptake of the polymer with bioactive agent is safe: studies have shown that the subject can metabolize/clear the polymer degradation products. These polymers and the invention intraocular polymer delivery compositions are, therefore, substantially non-inflammatory to the subject both at the site of injection and systemically, apart from the trauma caused by injection itself.

The biodegradable PEA, PEUR and PEU polymers useful in forming the invention biocompatible intraocular polymer particle and solid delivery compositions may contain multiple different α-amino acids in a single polymer molecule, for example, at least two different amino acids per repeat unit, or a single polymer molecule may contain multiple different α-amino acids in the polymer molecule, depending upon the size of the molecule. The polymer may also be a block co-polymer. In another embodiment, the polymer is used as one block in di- or tri-block copolymers, which are used to make micelles, as described below.

In addition, the polymers used in the invention polymer particle and solid delivery compositions display minimal hydrolytic degradation when tested in a saline (PBS) medium, but in an enzymatic solution, such as chymotrypsin or CT, a uniform erosive behavior has been observed.

Suitable protecting groups for use in the PEA, PEUR and PEU polymers include t- butyl or another as is known in the art. Suitable 1,4:3,6-dianhydrohexitols of general formula (II) include those derived from sugar alcohols, such as D-glucitol, D-mannitol, or L-iditol. Dianhydrosorbitol is the presently preferred bicyclic fragment of a 1,4:3,6-dianhydrohexitol for use in the PEA, PEUR and PEU polymers used in fabrication of the invention polymer particle delivery compositions.

The PEA, PEUR and PEU polymer molecules may also have an ophthalmologic or other bioactive agent attached thereto, optionally via a linker or incorporated into a crosslinker between molecules. For example, in one embodiment, the polymer is contained in a polymer-bioactive agent conjugate having structural formula (IX): 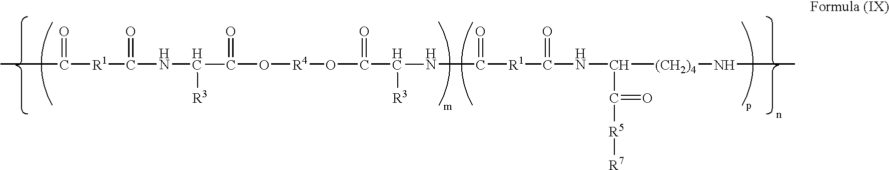
wherein n, m, p, R¹, R³, and R⁴ are as above, R⁵ is selected from the group consisting of —O—, —S— and —NR⁸—, wherein R⁸ is H or (C₁-C₈)alkyl; and R⁷ is the bioactive agent.

In yet another embodiment, two molecules of the polymer of structural formula (X) can be crosslinked to provide an —R⁵-R⁷-R⁵—conjugate. In another embodiment, as shown in structural formula (IX) below, the bioactive agent is covalently linked to two parts of a single polymer molecule of structural formula (IV) through the —R⁵-R⁷-R⁵— conjugate and R⁵ is independently selected from the group consisting of —O—, —S—, and —NR⁸—, wherein R⁸ is H or (C₁-C₈) alkyl; and R⁷ is the ophthalmologic or other bioactive agent.

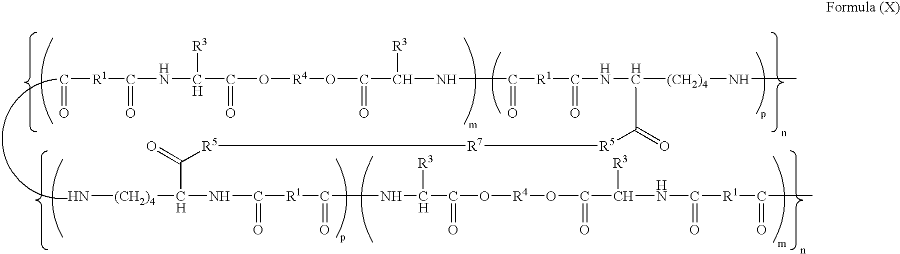

Alternatively still, as shown in structural formula (XI) below, a linker, -X-Y-, can be inserted between R⁵ and bioactive agent R⁷, in the molecule of structural formula (IV), wherein X is selected from the group consisting of (C₁-C₁₈) alkylene, substituted alkylene, (C₃-C₈) cycloalkylene, substituted cycloalkylene, 5-6 membered heterocyclic system-containing 1-3 heteroatoms selected from the group O, N, and S, substituted heterocyclic, (C₂-C₁₈) alkenyl, substituted alkenyl, alkynyl, substituted alkynyl, C₆ and C₁₀ aryl, substituted aryl, heteroaryl, substituted heteroaryl, alkylaryl, substituted alkylaryl, arylalkynyl, substituted arylalkynyl, arylalkenyl, substituted arylalkenyl, arylalkynyl, substituted arylalkynyl and wherein the substituents are selected from the group H, F, Cl, Br, I, (C₁-C₆) alkyl, —CN, —NO₂, —OH, —O(C₁-C₄) alkyl, —S(C₁-C₆) alkyl, —S[(═O)(C₁-C₆) alkyl], —S[(O₂)(C₁-C₆) alkyl], —C[(═O)(C₁-C₆) alkyl], CF₃, —O[(CO)—( C₁-C₆) alkyl], —S(O₂)[N(R⁹R¹⁰)], —NH[(C═O)(C₁-C₆) alkyl], —NH(C═O)N(R⁹R¹⁰), —N(R⁹R¹⁰); where R⁹ and R¹⁰ are independently H or (C₁-C₆) alkyl; and Y is selected from the group consisting of —O—, —S—, —S—S—, —S(O)—, —S(O₂)—, —NR⁸—, —C(═O)—, —OC(═O)—, —C(═O)O—, —OC(═O)NH—, —NR⁸C(═O)—, —C(═O)NR⁸—, —N R⁸C(═O)NR⁸—, —N R⁸C(═O)NR⁸—, and —NR⁸C(═S)N R⁸—.

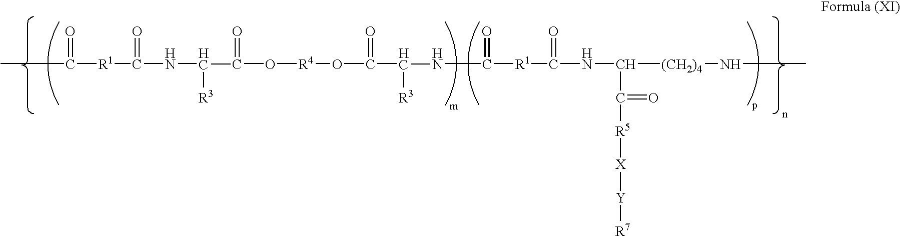

In another embodiment, two parts of a single macromolecule are covalently linked to the ophthalmologic or other bioactive agent through an —R⁵—R⁷—Y—X—R⁵— bridge (Formula XII): 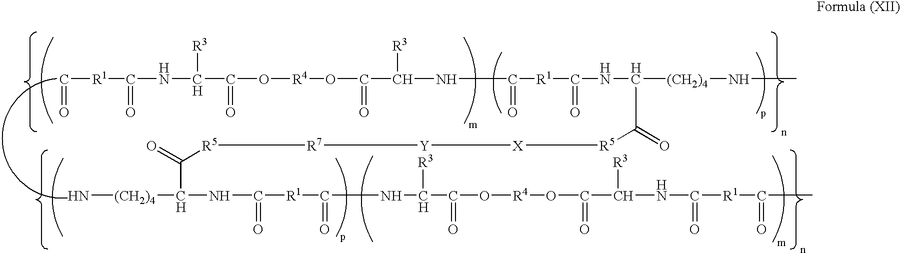
wherein, X is selected from the group consisting of (C₁-C₁₈) alkylene, substituted alkylene, (C₃-C₈) cycloalkylene, substituted cycloalkylene, 5-6 membered heterocyclic system containing 1-3 heteroatoms selected from the group O, N, and S, substituted heterocyclic, (C₂-C₁₈) alkenyl, substituted alkenyl, alkynyl, substituted alkynyl, (C₆-C₁₀) aryl, substituted aryl, heteroaryl, substituted heteroaryl, alkylaryl, substituted alkylaryl, arylalkynyl, substituted arylalkynyl, arylalkenyl, substituted arylalkenyl, arylalkynyl, substituted arylalkynyl, wherein the substituents are selected from the group consisting of H, F, Cl, Br, I, (C₁-C₆) alkyl, —CN, —NO_{2,}, —OH, —O(C₁-C₆) alkyl, —S(C₁-C₆) alkyl, —S[(═O)(C₁-C₆) alkyl], —S[(O₂)(C₁-C₆) alkyl], —C[(═O)(C₁-C₆) alkyl], CF₃, —O[(CO)—(C₁-C₆) alkyl], —S(O₂)[N(R⁹R¹⁰)], —NH[(C═O)(C₁-C₆) alkyl], —NH(C═O)N(R⁹R¹⁰), wherein R⁹ and R¹⁰ are independently H or (C₁-C₆) alkyl, and —N(R¹¹R¹²), wherein R¹¹ and R¹² are independently selected from (C₂-C₂₀) alkylene and (C₂-C₂₀) alkenylene.

In yet another embodiment, the intraocular polymer delivery composition contains four molecules of the polymer, except that only two of the four molecules omit R⁷ and are crosslinked to provide a single —R⁵—X—R⁵— conjugate.

The term “aryl” is used with reference to structural formulae herein to denote a phenyl radical or an ortho-fused bicyclic carbocyclic radical having about nine to ten ring atoms in which at least one ring is aromatic. In certain embodiments, one or more of the ring atoms can be substituted with one or more of nitro, cyano, halo, trifluoromethyl, or trifluoromethoxy. Examples of aryl include, but are not limited to, phenyl, naphthyl, and nitrophenyl.

The term “alkenylene” is used with reference to structural formulae herein to mean a divalent branched or unbranched hydrocarbon chain containing at least one unsaturated bond in the main chain or in a side chain.

The molecular weights and polydispersities herein are determined by gel permeation chromatography (GPC) using polystyrene standards. More particularly, number and weight average molecular weights (Mₙ and M_{w}) are determined, for example, using a Model 510 gel permeation chromatography (Water Associates, Inc., Milford, Mass.) equipped with a high-pressure liquid chromatographic pump, a Waters 486 UV detector and a Waters 2410 differential refractive index detector. Tetrahydrofuran (THF), N,N-dimethylformamide (DMF) or N,N-dimethylacetamide (DMAc) is used as the eluent (1.0 mL/min). Polystyrene or poly(methyl methacrylate) standards having narrow molecular weight distribution were used for calibration.

Methods for making polymers of structural formulas containing a α-amino acid in the general formula are well known in the art. For example, for the embodiment of the polymer of structural formula (I) wherein R⁴ is incorporated into an α-amino acid, for polymer synthesis the α-amino acid with pendant R³ can be converted through esterification into a bis-α, ω-diamine, for example, by condensing the α-amino acid containing pendant R³ with a diol HO—R⁴—OH. As a result, di-ester monomers with reactive α, ω-amino groups are formed. Then, the bis-α, ω-diamine is entered into a polycondensation reaction with a di-acid such as sebacic acid, or bis-activated esters, or bis-acyl chlorides, to obtain the final polymer having both ester and amide bonds (PEA). Alternatively, for example, for polymers of structure (I), instead of the di-acid, an activated di-acid derivative, e.g., bis-para-nitrophenyl diester, can be used as an activated di- acid. Additionally, a bis-di-carbonate, such as bis(p-nitrophenyl) dicarbonate, can be used as the activated species to obtain polymers containing a residue of a di-acid. In the case of PEUR polymers, a final polymer is obtained having both ester and urethane bonds.

More particularly, synthesis of the unsaturated poly(ester-amide)s (UPEAs) useful as biodegradable polymers of the structural formula (I) as disclosed above will be described, wherein 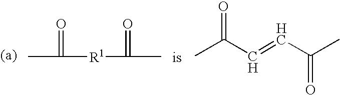
and/or (b) R⁴ is —CH₂—CH═CH—CH₂—. In cases where (a) is present and (b) is not present, R⁴ in (I) is —C₄H₈— or —C₆H₁₂—. In cases where (a) is not present and (b) is present, R¹ in (I) is —C₄H₈- or —C₈H₁₆—.

The UPEAs can be prepared by solution polycondensation of either (I) di-p-toluene sulfonic acid salt of bis(α-amino acid) di-ester of unsaturated diol and di-p-nitrophenyl ester of saturated dicarboxylic acid or (2) di-p-toluene sulfonic acid salt of bis (α-amino acid) diester of saturated diol and di-nitrophenyl ester of unsaturated dicarboxylic acid or (3) di-p-toluene sulfonic acid salt of bis(α-amino acid) diester of unsaturated diol and di-nitrophenyl ester of unsaturated dicarboxylic acid.

The aryl sulfonic acid salts of diamines are known for use in synthesizing polymers containing amino acid residues. The p-toluene sulfonic acid salts are used instead of the free diamines because the aryl sulfonic salts of bis (α-amino acid) diesters are easily purified through recrystallization and render the amino groups as less reactive ammonium tosylates throughout workup. In the polycondensation reaction, the nucleophilic amino group is readily revealed through the addition of an organic base, such as triethylamine, reacts with bis-electrophilic monomer, so the polymer product is obtained in high yield.

Bis-electrophilic monomers, for example, the di-p-nitrophenyl esters of unsaturated dicarboxylic acid, can be synthesized from p-nitrophenyl and unsaturated dicarboxylic acid chloride, e.g., by dissolving triethylamine and p-nitrophenol in acetone and adding unsaturated dicarboxylic acid chloride dropwise with stirring at −78° C. and pouring into water to precipitate product. Suitable acid chlorides included fumaric, maleic, mesaconic, citraconic, glutaconic, itaconic, ethenyl-butane dioic and 2-propenyl-butanedioic acid chlorides. For polymers of structure (V) and (VI), bis-p-nitrophenyl dicarbonates of saturated or unsaturated diols are used as the activated monomer. Dicarbonate monomers of general structure (XIII) are employed for polymers of structural formula (V) and (VI), 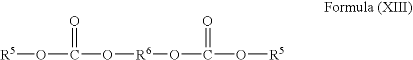
wherein each R⁵ is independently (C₆-C₁₀) aryl optionally substituted with one or more nitro, cyano, halo, trifluoromethyl, or trifluoromethoxy; and R⁶ is independently (C₂-C₂₀) alkylene or (C₂-C20) alkyloxy, or (C₂-C₂₀) alkenylene.

Suitable therapeutic diol compounds that can be used to prepare bis(α-amino acid) diesters of therapeutic diol monomers, or bis(carbonate) of therapeutic di-acid monomers, for introduction into the invention intraocular polymer delivery compositions include naturally occurring therapeutic diols, such as 17-β-estradiol, a natural and endogenous hormone, useful in preventing restenosis and tumor growth. The procedure for incorporation of a therapeutic diol, such as an ophthalmologic diol as disclosed herein, into the backbone of a PEA, PEUR or PEU polymer is illustrated in this application by Example 8, in which active steroid hormone 17-β-estradiol containing mixed hydroxyls—secondary and phenolic—is introduced into the backbone of a PEA polymer. When the PEA polymer is used to fabricate particles or solids and the particles or solids are implanted intraocularly, the therapeutic diol is released from the particles or solids at a controlled rate. In the present invention, the preferred therapeutic diol for incorporation into the backbone of the polymer is an ophthalmologic diol.

Due to the versatility of the PEA, PEUR and PEU polymers used in the invention compositions, the amount of the therapeutic diol or di-acid incorporated in a polymer backbone can be controlled by varying the proportions of the building blocks of the polymer. For example, depending on the composition of the PEA, loading of up to 40% w/w of 17β-estradiol can be achieved. Two different regular, linear PEAs with various loading ratios of 17β-estradiol illustrate this concept in Scheme I below: 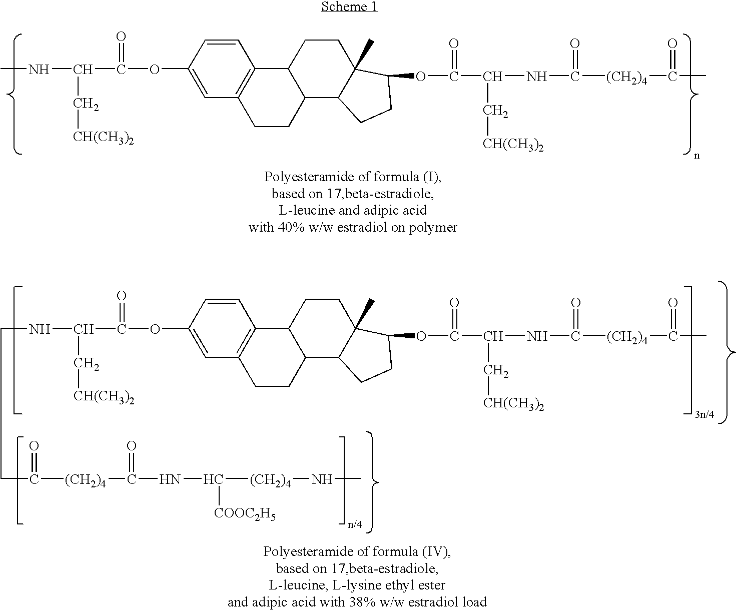
Similarly, the loading of the therapeutic diol into PEUR and PEU polymer can be varied by varying the amount of two or more building blocks of the polymer.

Suitable ophthalmologic synthetic steroid based diols, based on testosterone or cholesterol, that can be dispersed in or incorporated into the backbones of the polymers used in the invention intraocular implant compositions include such compounds a hydrocortisone, fluorocortisone, cortisone, aldosterone, betamethasone, prednisolone, dexamethasone, fluocinolone acetonide, and the like.

Additional ophthalmologic diol compounds that can be used to prepare an amide linkage in the PEA polymer compositions of the invention include, for example, latanoprost, brimatoprost, travoprost, cidofovir, pencyclovir, and the like.

Additional, synthetic steroid based therapeutic diols based on testosterone or cholesterol, such as 4-androstene-3, 17 diol (4-androstenediol), 5-androstene-3, 17 diol (5-androstenediol), 19-nor5-androstene-3, 17 diol (19-norandrostenediol) are also suitable for incorporation into the backbone of PEA. PEUR and PEU polymers according to this invention. For example, therapeutic diol compounds suitable for use in preparation of the invention intraocular polymer particle or solid delivery compositions include, for example, amikacin; amphotericin B; apicycline; apramycin; arbekacin; azidamfenicol; bambermycin(s); butirosin; carbomycin; cefpiramide; chloramphenicol; chlortetracycline; clindamycin; clomocycline; demeclocycline; diathymosulfone; dibekacin, dihydrostreptomycin; dirithromycin; doxycycline; erythromycin; fortimicin(s); gentamycin(s); glucosulfone solasulfone; guamecycline; isepamicin; josamycin; kanamycin(s); leucomycin(s); lincomycin; lucensomycin; lymecycline; meclocycline; methacycline; micronomycin; midecamycin(s); minocycline; mupirocin; natamycin; neomycin; netilmicin; oleandomycin; oxytetracycline; paromycin; pipacycline; podophyllinic acid 2-ethylhydrazine; priniycin; ribostamycin; rifamide; rifampin; rafamycin SV; rifapentine; rifaximin; ristocetin; rokitamycin; rolitetracycline; rasaramycin; roxithromycin; sancycline; sisomicin; spectinomycin; spiramycin; streptomycin; teicoplanin; tetracycline; thiamphenicol; theiostrepton; tobramycin; trospectomycin; tuberactinomycin; vancomycin; candicidin(s); chlorphenesin; dermostatin(s); filipin; fungichromin; kanamycin(s); leucomycins(s); lincomycin; lvcensomycin; lymecycline; meclocycline; methacycline; micronomycin; midecamycin(s); minocycline; mupirocin; natamycin; neomycin; netilmicin; oleandomycin; oxytetracycline; paramomycin; pipacycline; podophyllinic acid 2-ethylhydrazine; priycin; ribostamydin; rifamide; rifampin; rifamycin SV; rifapentine; rifaximin; ristocetin; rokitamycin; rolitetracycline; rosaramycin; roxithromycin; sancycline; sisomicin; spectinomycin; spiramycin; strepton; otbramycin; trospectomycin; tuberactinomycin; vancomycin; candicidin(s); chlorphenesin; dermostatin(s); filipin; fungichromin; meparticin; mystatin; oligomycin(s); erimycinA; tubercidin; 6-azauridine; aclacinomycin(s); ancitabine; anthramycin; azacitadine; bleomycin(s) carubicin; carzinophillin A; chlorozotocin; chromomcin(s); doxifluridine; enocitabine; epirubicin; gemcitabine; mannomustine; menogaril; atorvasi pravastatin; clarithromycin; leuproline; paclitaxel; mitobronitol; mitolactol; mopidamol; nogalamycin; olivomycin(s); peplomycin; pirarubicin; prednimustine; puromycin; ranimustine; tubercidin; vinesine; zorubicin; coumetarol; dicoumarol; ethyl biscoumacetate; ethylidine dicoumarol; iloprost; taprostene; tioclomarol; amiprilose; romurtide; sirolimus (rapamycin); tacrolimus; salicyl alcohol; bromosaligenin; ditazol; fepradinol; gentisic acid; glucamethacin; olsalazine; S-adenosylmethionine; azithromycin; salmeterol; budesonide; albuteal; indinavir; fluvastatin; streptozocin; doxorubicin; daunorubicin; plicamycin; idarubicin; pentostatin; metoxantrone; cytarabine; fludarabine phosphate; floxuridine; cladriine; capecitabien; docetaxel; etoposide; topotecan; vinblastine; teniposide, and the like. The therapeutic diol can be selected to be either a saturated or an unsaturated diol.

Suitable naturally occurring and synthetic therapeutic di-acids that can be used to prepare an amide linkage in the PEA polymer compositions of the invention include, for example, bambermycin(s); benazepril; carbenicillin; carzinophillin A; cefixime; cefininox cefpimizole; cefodizime; cefonicid; ceforanide; cefotetan; ceftazidime; ceftibuten; cephalosporin C; cilastatin; denopterin; edatrexate; enalapril; lisinopril; methotrexate; moxalactam; nifedipine; olsalazine; penicillin N; ramipril; quinacillin; quinapril; temocillin; ticarcillin; Tomudex® (N-[[5-[[(1,4-Dihydro-2-methyl-4-oxo-6-guinazolinyl)methyl]methylamino]-2-thienyl]carbonyl]-L-glutamic acid), and the like. The safety profile of naturally occurring therapeutic di-acids is believed to surpass that of synthetic therapeutic di-acids. The therapeutic di-acid can be either a saturated or an unsaturated di-acid.

The chemical and therapeutic properties of the above described ophthalmologic and other therapeutic diols and di-acids as inhibitors of macular degeneration, tumor inhibitors, cytotoxic antimetabolites, antibiotics, and the like, are well known in the art and detailed descriptions thereof can be found, for example, in the 13th Edition of The Merck Index (Whitehouse Station, N.J., USA).

The di-aryl sulfonic acid salts of diesters of α-amino acid and unsaturated diol can be prepared by admixing α-amino acid, e.g., p-aryl sulfonic acid monohydrate and saturated or unsaturated diol in toluene, heating to reflux temperature, until water evolution is minimal, then cooling. The unsaturated diols include, for example, 2-butene-1,3-diol and 1,18-octadec-9-en-diol.

Saturated di-p-nitrophenyl esters of dicarboxylic acid and saturated di-p-toluene sulfonic acid salts of bis-α-amino acid esters can be prepared as described in U.S. Pat. No. 6,503,538 B1.

Synthesis of the unsaturated poly(ester-amide)s (UPEAs) useful as biodegradable polymers of the structural formula (I) as disclosed above will now be described. UPEAs having the structural formula (I) can be made in similar fashion to the compound (VII) of U. S. Pat. No. 6,503,538 B1, except that R⁴ of (III) of U.S. Pat. No. 6,503,538 and/or R¹ of (V) of 6,503,538 is (C₂-C₂₀) alkenylene as described above. The reaction is carried out, for example, by adding dry triethylamine to a mixture of said (III) and (IV) of U.S. Pat. No. 6,503,538 and said (V) of U.S. Pat. No. 6,503,538 in dry N,N-dimethylacetamide, at room temperature, then increasing the temperature to 80° C. and stirring for 16 hours, then cooling the reaction solution to room temperature, diluting with ethanol, pouring into water, separating polymer, washing separated polymer with water, drying to about 30° C. under reduced pressure and then purifying up to negative test on p-nitrophenol and p-toluene sulfonate. A preferred reactant (IV) of U.S. Pat. No. 6,503,538 is p-toluene sulfonic acid salt of Lysine benzyl ester, the benzyl ester protecting group is preferably removed from (II) to confer biodegradability, but it should not be removed by hydrogenolysis as in Example 22 of U.S. Pat. No. 6,503,538 because hydrogenolysis would saturate the desired double bonds; rather the benzyl ester group should be converted to an acid group by a method that would preserve unsaturation. Alternatively, the lysine reactant (IV) of U.S. Pat. No. 6,503,538 can be protected by a protecting group different from benzyl that can be readily removed in the finished product while preserving unsaturation, e.g., the lysine reactant can be protected with t-butyl (i.e., the reactant can be t-butyl ester of lysine) and the t-butyl can be converted to H while preserving unsaturation by treatment of the product (II) with acid.

A working example of the compound having structural formula (I) is provided by substituting p-toluene sulfonic acid salt of bis(L-phenylalanine) 2-butene-1,4-diester for (III) in Example 1 of U.S. Pat. No. 6,503,538 or by substituting di-p-nitrophenyl fumarate for (V) in Example I of U.S. Pat. No. 6,503,538 or by substituting the p-toluene sulfonic acid salt of bis(L-phenylalanine) 2-butene-1,4-diester for III in Example 1 of U.S. Pat. No. 6,503,538 and also substituting bis-p-nitrophenyl fumarate for (V) in Example 1 of U.S. Pat. No. 6,503,538.

In unsaturated compounds having either structural formula (I) or (IV), the following hold. An amino substituted aminoxyl (N-oxide) radical bearing group, e.g., 4-amino TEMPO, can be attached using carbonyldiimidazol, or suitable carbodiimide, as a condensing agent. Bioactive agents, as described herein, can be attached via the double bond functionality. Hydrophilicity can be imparted by bonding to poly(ethylene glycol) diacrylate.

In yet another aspect, the PEA and PEUR polymers contemplated for use in forming the invention polymer particle delivery systems include those set forth in U.S. Pat. Nos. 5,516,881; 6,476,204; 6,503,538; and in U.S. application Ser. Nos. 10/096,435; 10/101,408; 10/143,572; and 10/194,965; the entire contents of each of which is incorporated herein by reference.

The biodegradable PEA, PEUR and PEU polymers can contain from one to multiple different ophthalmologic compounds and α-amino acids per polymer molecule and preferably have weight average molecular weights ranging from 10,000 to 125,000; these polymers and copolymers typically have intrinsic viscosities at 25° C., as determined by standard viscosimetric methods, ranging from 0.3 to 4.0, for example, ranging from 0.5 to 3.5.

PEA and PEUR polymers contemplated for use in the practice of the invention can be synthesized by a variety of methods well known in the art. For example, tributyltin (IV) catalysts are commonly used to form polyesters such as poly(ε-caprolactone), poly(glycolide), poly(lactide), and the like. However, it is understood that a wide variety of catalysts can be used to form polymers suitable for use in the practice of the invention.

Such poly(caprolactones) contemplated for use have an exemplary structural formula (XIV) as follows:

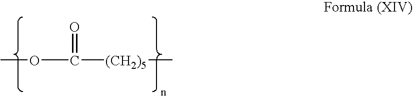

Poly(glycolides) contemplated for use have an exemplary structural formula (XV) as follows:

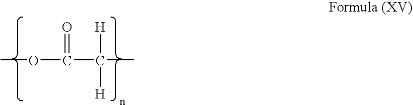

Poly(lactides) contemplated for use have an exemplary structural formula (XVI) as follows:

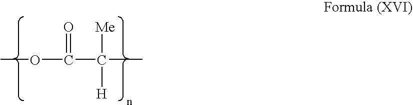

An exemplary synthesis of a suitable poly(lactide-co-ε-aprolactone) including an aminoxyl moiety is set forth as follows. The first step involves the copolymerization of lactide and □-caprolactone in the presence of benzyl alcohol using stannous octoate as the catalyst to form a polymer of structural formula (XVII).

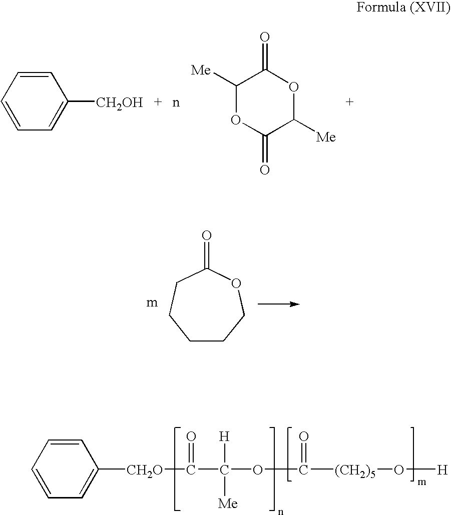

The hydroxy terminated polymer chains can then be capped with maleic anhydride to form polymer chains having structural formula (XVIII):

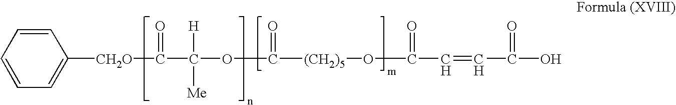

At this point, 4-amino-2,2,6,6-tetramethylpiperidine-1-oxy can be reacted with the carboxylic end group to covalently attach the aminoxyl moiety to the copolymer via the amide bond which results from the reaction between the 4-amino group and the carboxylic acid end group. Alternatively, the maleic acid capped copolymer can be grafted with polyacrylic acid to provide additional carboxylic acid moieties for subsequent attachment of further aminoxyl groups.

In unsaturated compounds having structural formula (VII) for PEU, the following hold: An amino substituted aminoxyl (N-oxide) radical bearing group e.g., 4-amino TEMPO, can be attached using carbonyldiimidazole, or suitable carbodiimide, as a condensing agent. Additional bioactive agents, and the like, as described herein, optionally can be attached via the double bond functionality provided that the therapeutic diol residue in the polymer composition does not contain a double or triple bond.

For example, the invention high molecular weight semi-crystalline PEUs having structural formula (VII) can be prepared inter-facially by using phosgene as a bis-electrophilic monomer in a chloroform/water system, as shown in the reaction scheme (2) below: 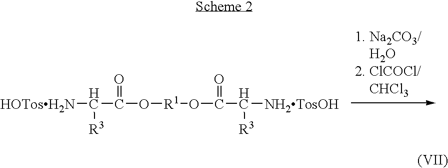
Synthesis of copoly(ester ureas) (PEUs) containing L-Lysine esters and having structural formula (VII) can be carried out by a similar scheme (3): 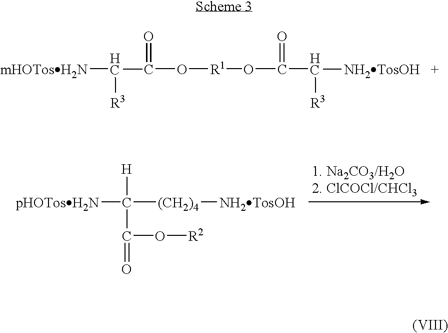
A 20% solution of phosgene (CICOCI) (highly toxic) in toluene, for example (commercially available (Fluka Chemie, GMBH, Buchs, Switzerland), can be substituted either by diphosgene (trichloromethylchloroformate) or triphosgene (bis(trichloromethyl)carbonate). Less toxic carbonyldiimidazole can be also used as a bis-electrophilic monomer instead of phosgene, di-phosgene, or tri-phosgene.

General Procedure for Synthesis of PEUs It is necessary to use cooled solutions of monomers to obtain PEUs of high molecular weight. For example, to a suspension of di-p-toluenesulfonic acid salt of bis(a-amino acid)-α,ω-alkylene diester in 150 mL of water, anhydrous sodium carbonate is added, stirred at room temperature for about 30 minutes and cooled to about 2-0° C., forming a first solution. In parallel, a second solution of phosgene in chloroform is cooled to about 15-10° C. The first solution is placed into a reactor for interfacial polycondensation and the second solution is quickly added at once and stirred briskly for about 15 min. Then chloroform layer can be separated, dried over anhydrous Na₂SO₄, and filtered. The obtained solution can be stored for further use.

All the exemplary PEU polymers fabricated were obtained as solutions in chloroform and these solutions are stable during storage. However, some polymers, for example, 1-Phe-4, become insoluble in chloroform after separation. To overcome this problem, polymers can be separated from chloroform solution by casting onto a smooth hydrophobic surface and allowing chloroform to evaporate to dryness. No further purification of obtained PEUs is needed. The yield and characteristics of exemplary PEUs obtained by this procedure are summarized in Table 1 herein.

General Procedure for Preparation of porous PEUs. Methods for making the PEU polymers containing α-amino acids in the general formula will now be described. For example, for the embodiment of the polymer of formula (I) or (III), the α-amino acid can be converted into a bis(α-amino acid)-α,ω-diol-diester monomer, for example, by condensing the α-amino acid with a diol HO—R¹—OH. As a result, ester bonds are formed. Then, acid chloride of carbonic acid (phosgene, diphosgene, triphosgene) is entered into a polycondensation reaction with a di-p-toluenesulfonic acid salt of a bis(α-amino acid) -alkylene diester to obtain the final polymer having both ester and urea bonds. In the present invention, at least one therapeutic diol can be used in the polycondensation protocol.

The unsaturated PEUs can be prepared by interfacial solution condensation of di-p-toluenesulfonate salts of bis(α-amino acid)-alkylene diesters, comprising at least one double bond in R¹. Unsaturated diols useful for this purpose include, for example, 2-butene-1,4-diol and 1,18-octadec-9-en-diol. Unsaturated monomer can be dissolved prior to the reaction in alkaline water solution, e.g. sodium hydroxide solution. The water solution can then be agitated intensely, under external cooling, with an organic solvent layer, for example chloroform, which contains an equimolar amount of monomeric, dimeric or trimeric phosgene. An exothermic reaction proceeds rapidly, and yields a polymer that (in most cases) remains dissolved in the organic solvent. The organic layer can be washed several times with water, dried with anhydrous sodium sulfate, filtered, and evaporated. Unsaturated PEUs with a yield of about 75%-85% can be dried in vacuum, for example at about 45° C.

To obtain a porous, strong material, L-Leu based PEUs, such as 1-L-Leu-4 and 1-L-Leu-6, both of formula (VII), can be fabricated using the general procedure described below.

Such procedure is less successful in formation of a porous, strong material when applied to L-Phe based PEUs.

The reaction solution or emulsion (about 100 mL) of PEU in chloroform, as obtained just after interfacial polycondensation, is added dropwise with stirring to 1,000 mL of about 80-85° C. water in a glass beaker, preferably a beaker made hydrophobic with dimethyldichlorsilane to reduce the adhesion of PEU to the beaker's walls. The polymer solution is broken in water into small drops and chloroform evaporates rather vigorously. Gradually, as chloroform is evaporated, small drops combine into a compact tar-like mass that is transformed into a sticky rubbery product. This rubbery product is removed from the beaker and put into hydrophobized cylindrical glass-test-tube, which is thermostatically controlled at about 80° C. for about 24 hours. Then the test-tube is removed from the thermostat, cooled to room temperature, and broken to obtain the polymer. The obtained porous bar is placed into a vacuum drier and dried under reduced pressure at about 80° C. for about 24 hours. In addition, any procedure known in the art for obtaining porous polymeric materials can also be used.

Properties of high-molecular-weight porous PEUs made by the above procedure yielded results as summarized in Table 2.

TABLE 1
Properties of PEU Polymers of Formula (VII) and (VIII)

| | Yield | η_{red}^{a)} | | | | Tg^{c)} | Tₘc) |
|---|---|---|---|---|---|---|---|
| PEU* | [%] | [dL/g] | M_{w}^{b)} | Mₙ^{b)} | M_{w}/Mₙ^{b)} | [° C.] | [° C.] |
| 1-L-Leu-4 | 80 | 0.49 | 84000 | 45000 | 1.90 | 67 | 103 |
| 1-L-Leu-6 | 82 | 0.59 | 96700 | 50000 | 1.90 | 64 | 126 |
| 1-L-Phe-6 | 77 | 0.43 | 60400 | 34500 | 1.75 | — | 167 |
| [1-L-Leu-6]_{0.75}- [1-L- | 84 | 0.31 | 64400 | 43000 | 1.47 | 34 | 114 |
| Lys(OBn)]_{0.25} | | | | | | | |
| 1-L-Leu-DAS | 57 | 0.28 | 55700^{d)} | 27700^{d)} | 2.1^{d)} | 56 | 165 |

*PEUs of general formula (VII), where,
1-L-Leu-4: R⁴ = (CH₂)₄, R³ = i-C₄H₉
1-L-Leu-6: R⁴ = (CH₂)₆, R³ = i-C₄H₉
1-L-Phe-6: .R⁴ = (CH₂)₆, R³ = -CH₂-C₆H₅.
1-L-Leu-DAS: R⁴ = 1,4:3,6-dianhydrosorbitol, R³ = i-C₄H
^{a)Reduced viscosities were measured in DMF at 25° C. and a concentration 0.5 g/dL}
^{b)GPC Measurements were carried out in DMF, (PMMA)}
^{c)Tg taken from second heating curve from DSC Measurements (heating rate 10° C./min).}
^{d)GPC Measurements were carried out in DMAc, (PS)}

Tensile strength of illustrative synthesized PEUs was measured and results are summarized in Table 2. Tensile strength measurement was obtained using dumbbell-shaped PEU films (4×1.6 cm), which were cast from chloroform solution with average thickness of 0.125 mm and subjected to tensile testing on tensile strength machine (Chatillon TDC200) integrated with a PC using Nexygen FM software (Amtek, Largo, Fla.) at a crosshead speed of 60 mm/min. Examples illustrated herein can be expected to have the following mechanical properties:
1. A glass transition temperature in the range from about 30° C. to about 90° C., for. example, in the range from about 35° C. to about 70° C.;
2. A film of the polymer with average thickness of about 1.6 cm will have tensile stress at yield of about 20 Mpa to about 150 Mpa, for example, about 25 Mpa to about 60 Mpa;
3. A film of the polymer with average thickness of about 1.6 cm will have a percent elongation of about 10% to about 200%, for example about 50% to about 150%; and

4. A film of the polymer with average thickness of about 1.6 cm will have a Young's modulus in the range from about 500 MPa to about 2000 MPa. Table 2 below summarizes the properties of exemplary PEUs of this type.

TABLE 2
Mechanical Properties of PEUs

| | | Tensile | | |
|---|---|---|---|---|
| | | Stress | Percent | Young's |
| Polymer | Tg^{a)} | at Yield | Elongation | Modulus |
| designation | (° C.) | (MPa) | (%) | (MPa) |
| 1-L-Leu-6 | 64 | 21 | 114 | 622 |
| [1-L-Leu-6]_{0.75} − | 34 | 25 | 159 | 915 |
| [1-L-Lys(OBn)]_{0.25} | | | | |

In another embodiment, the invention provides solid polymer intraocular delivery compositions comprising one or more solid layers comprising at least one biodegradable, biocompatible polymer as a carrier layer into which is dispersed, mixed, dissolved, homogenized, or matrixed (i.e., “dispersed”) at least one ophthalmologic agent. Two or more ophthalmologic agents may also be dispersed into a carrier layer, or invention compositions having more than one carrier layer may have two or more ophthalmologic agents dispersed into separate carrier layers therein.

In one embodiment, the invention provides a solid polymer intraocular delivery composition for implant intraocularly, said composition comprising at least one biodegradable, biocompatible polymer having a chemical formula described by general structural formulas (I)-(IV-VIII) as described herein into which is dispersed at least one ophthalmologic agent for release at a controlled rate over a considerable period of time, for example, over a period of three months to about twelve months. Optionally, an additional bioactive agent, as described herein, may also be dispersed in the at least one polymer. The ophthalmologic agent and any optional bioactive agent present therein is released from the composition in situ (i.e., intraocularly) as a result of biodegradation of the various polymer layers in the composition.

The solid polymer intraocular delivery composition may further comprise at least one coating layer of a biodegradable, biocompatible polymer, which may or may not have dispersed therein such an ophthalmologic agent. The purpose of the coating layer of polymer, for example a pure polymer shell, is to slow release of the ophthalmologic agent contained in the composition. The PEA, PEUR and PEU polymers of formulas (I) and (IV-VIII) described herein readily absorb water, allowing hydrophilic molecules to readily diffuse therethrough. This characteristic makes such PEA, PEUR and PEU polymers suitable for use as a coating on the invention solid polymer compositions to control release rate of the at least one bioactive agent therefrom.

Rate of release of the at least one ophthalmologic agent from the invention solid polymer intraocular delivery compositions can be controlled, not only by selection of the polymers in various layers of the composition, but by adjusting the coating thickness and density, as well as by the number of coating layers contained in the invention composition. Density of the coating layer can be adjusted by adjusting loading of the active agent(s) in the coating layer. For example, when the coating layer contains no bioactive agent, the polymer coating layer is densest, and an ophthalmological, or optional bioactive, agent from the interior of the composition elutes through the coating layer most slowly. By contrast, when an ophthalmologicall, or optional bioactive, agent is dispersed within (i.e. is mixed or “matrixed” with) biodegradable, biocompatible polymer in the coating layer, the coating layer becomes porous once any active agent in the coating layer has eluted out, starting from the outer surface of the coating layer. A porous coating layer once formed by this process, an ophthalmologic agent in the carrier layer(s) of the solid composition can elute at an increased rate. The higher the active agent loading in the coating layer, the lower the density of the coating layer and the higher the elution rate. Although loading of active agent in the coating layer can be lower or higher than that in the carrier layer(s), for slowest sustained delivery of an ophthalmologic agent at a controlled rate, the coating layer is a pure polymer shell. There may be multiple coating layers as well as multiple carrier layers in the invention composition.

Accordingly, in one embodiment the invention provides a solid polymer composition for controlled release of an ophthalmologic agent, said composition comprising a carrier layer containing at least one ophthalmologic agent dispersed in a biodegradable, biocompatible polymer having a structural formula described by structural formulas (I) and (IV-VIII), and at least one coating layer that covers the carrier layer, wherein the coating layer comprises a biodegradable, biocompatible polymer, such as those described by structural formulas (I) and (IV). For convenience in manufacture, the polymer of the coating layer may be the same as the polymer of the carrier layer.

However, it has been discovered that, during manufacture, solvent in the polymer dispersion used to make the coating layer(s) of the invention solid polymer intraocular delivery composition tends to elute a matrixed ophthalmologic agent out of the carrier layer, even though the carrier layer has been dried prior to application of the coating layer. To prevent the solvent used in applying the coating layer from robbing the carrier layer of its load of bioactive agent, the invention composition may further comprise a barrier layer between a carrier layer and each of one or more coating layers. The barrier layer is made using a liquid polymer that will not dissolve in the solvent used in the polymer solution or dispersion that lays down the coating layer in the invention composition, but which barrier layer polymer dissolves in physiologic conditions, for example in the presence of aqueous conditions and physiologic enzymes. Thus, the barrier layer(s), as well as the coating layer(s) of the invention composition, aid in controlling the release rate of the ophthalmologic agent from the carrier polymer layer.

In certain embodiments, invention solid polymer intraocular delivery compositions can have one or multiple sets of the barrier layer and coating layer, with the coating layer being exterior in the final composition to each successive set. Consequently, the carrier layer is sequestered within the protective sets of barrier layer and coating layer. For example, one to ten sets of the barrier layer and coating layer may be present, e.g., one to eight sets, with the number of sets determining the rate of release of the ophthalmologic agent in the carrier layer from the composition. A larger number of sets provide for a slower release rate and a lesser number of sets provides for a faster release rate.

In one embodiment, the sets of barrier layer and coating layer may be aligned in a parallel configuration on either side of the carrier layer in a sandwich structure, with the carrier layer at the center of two sets of barrier layer and coating layer on either side. In another embodiment, successive outwardly lying layers of the multiple sets of layers encompass all interior layers to form an onionskin structure with the carrier layer sequestered at the center of the composition. Thickness of the carrier layer and concentration of the one or more ophthalmologic agents (and optional bioactive agents) in the carrier layer determine total dosage of each agent that the composition can deliver when implanted. After implant or in vitro exposure to physiological conditions (e.g. water and enzymes) the outward most layers of coating and barrier layer begin to biodegrade as encountered and the matrixed bioactive agent(s) elutes from the sequestered carrier layer. As the concentration of bioactive agent in the interior carrier layer diminishes, the surface area of the composition will tend to diminish as well due to biodegradation, with both effects tending to slow the rate of release of the bioactive agent in a controlled manner.

In another embodiment, the invention composition comprises multiple sets of carrier and barrier layers arranged in an onionskin structure with a single exterior coating of pure biodegradable, biocompatible polymer or alternating layers of barrier layer and coating as described above. In this embodiment, water and enzymes from the environment, e.g., intraocular tissue surroundings, successively dissolve the outward most coating(s) and barrier layer(s) encountered to elute ophthalmologic agent from successive carrier layers, thereby releasing the ophthalmologic agent(s), which can either be matrixed in the polymer or incorporated into the polymer backbone. If the concentration of ophthalmologic active agent is substantially constant in the multiple layers of the onionskin structure, the rate of delivery will diminish in proportion as the surface area of the onionskin structure diminishes. To accomplish a more constant rate of delivery of the matrixed bioactive agent(s), it is recommended in this embodiment to utilize a gradient of concentration of bioactive agent in the carrier layers in the onionskin structure, with a higher concentration being used in inner carrier layers in proportion to the diminishing surface area of the composition. Those skilled in the art will understand that routine principles of fluid dynamics can be used to calculate the theoretical rate of delivery and to obtain a substantially constant rate of delivery as the onionskin structure diminishes in surface area, if desired.

In yet another embodiment, the invention solid polymer intraocular delivery composition comprises a carrier layer with additional polymer layers arranged in an onionskin or sandwich structure about the carrier layer. In one alternative, there can be a single exterior coating layer of pure biodegradable, biocompatible polymer formed by spraying the carrier layer and coating layer, for example, as an aerosol. This structure favored for compositions having a thickness of 0.1 mm to 2.5 mm in thickness for rapid release of the ophthalmologic agent(s). In another alternative, several coating layers extend outwardly from the core carrier layer in an onionskin or sandwich structure. Alternatively still, to achieve a more constant rate of delivery, multiple carrier layers can be employed, provided that each carrier layer is subsequently coated with a coating layer. To effect a gradient of concentration of bioactive molecule(s) in the carrier layer(s) in the onionskin structure, a higher concentration of ophthalmologic molecule(s) is used in inner carrier layers in proportion to the outer carrier layers, such that the rate of release of ophthalmologic molecule(s) can be maintained as the surface area of the composition diminishes during the biodegradation of the invention composition.

The invention solid intraocular polymer delivery composition typically has a three-dimensional shape compatible with the properties of the polymer and which meets the therapeutic and delivery requirements for a given ophthalmologic agent and route of administration., such as a wafer, sheet or film, ball, disc, cylinder, fiber, tube, and the like. Constructs will be sized according to delivery requirements and location of administration. For example, for subconjunctival administration, the size of the construct will be preferably no larger than about a 1 mm by 1 mm by 7 mm rectangle or cylinder suitable for injection through a hypodermic needle. The bore requirements of the hypodermic needle should coincide with the route and location of administration, e.g., for subconjunctival administration the needle bore should be about 18 to about 25 gauge.

The invention solid intraocular polymer delivery compositions may optionally additionally comprise (e.g., be fabricated to include a means to secure the solid composition to ocular tissue at the point of administration, such as a suture tab, to prevent migration of the construct to other locations that are not within the intended route of administration.

Invention solid polymer intraocular delivery compositions will be capable of releasing the ophthalmologic agent over a range from about twenty-four hours, about seven days, about thirty days, or about ninety days, or longer, for example up to three years, depending upon the condition whose treatment requires release of the ophthalmologic agent. In one embodiment the range is from about 1-2 days up to about six months. For example, for delivery of ophthalmologic agent to the back of the eye (e.g., to treat AMD) the invention solid composition will release an effective amount of the suitable ophthalmologic agent over about six months.

The invention solid compositions can have the ophthalmologic agent present in a concentration in the range from about 0.1% up to 99.9% by weight of the composition. In addition the invention solid compositions can withstand end stage sterilization by at least one method, preferably by multiple methods, e.g., steam, gamma radiation, e-radiated, and the like.

In one embodiment, the invention solid polymer intraocular delivery compositions can be physically and chemically stable for a minimum of two years at 5° C., preferably at 25° C. (room temperature).

Preferred solid shapes are discs and cylinders; however, any convenient three dimensional shape can be used, such as a disc, sheet, film, fiber or tube. Those of skill in the art of fluid dynamics will understand that choice of the shape of the solid composition will also affect the rate of elution of bioactive agent from the invention composition. Since, the implant may be placed during surgery, a cylinder or rectangle sized to fit down the interior bore of a hypodermic needle or pharmaceutical delivery needle may be suitable for placement during the surgery. In general, , the invention composition for interior delivery will have dimensions no larger than about 1 mm by 1 mm by about 7 mm. For topical application, however, the size may be larger. For example, if a sheet is used, the sheet may have any size suitable for application to the surface of the eye, for example, about the dimensions of the exterior of the eye, or about 25 mm×25 mm.

In yet further embodiments, the invention solid polymer intraocular delivery compositions are porous solids. A “porous solid” fabrication of the invention polymer compositions, as the term is used herein, means compositions that have a ratio of surface area to volume greater than 1:1. As described below, the maximum porosity of an invention solid polymer composition will depend upon its shape and method of fabrication. Any of the various methods for creating pores or “scaffolding” for cell growth in polymers may be used in connection with the present invention. The following examples of methods for fabricating the invention compositions as porous solids are illustrative and not intended to be limiting.

In the first example, porosity of the composition is achieved after the solid polymer delivery composition is formed by cutting pores through the layers of the composition, for example by laser cutting or etching, such as reactive ion etching,. For example, short-wavelength UV laser energy is superior to etching for clean-cutting, drilling, and shaping the invention polymer composition. UV laser technology first developed by Massachusetts Institute of Technology (MIT)allows for removal of very fine and measured amounts of material as a plasma plume by “photo-ablation” with each laser pulse, leaving a cleanly-sculpted pore, or channel. The large size characteristic of the UV excimer laser beam allows it to be separated into multiple beamlets through near-field imaging techniques, so that multiple pores, for example, can be simultaneously bored with each laser pulse. Imaging techniques also allow sub-micron resolution so that nano features can be effectively controlled and shaped. For example, micro-machining of scaffold thickness of 250 microns and channel depth of 200 microns, with pore depth of 50 microns has been achieved using this technique on Polycarbonate, Polyethylene Terephthalate, and Polyimide.

In another example, porosity of the invention solid polymer intraocular delivery composition is achieved by adding a pore-forming substance, such as a gas, or a pore-forming substance (i.e., a porogen) that releases a gas when exposed to heat or moisture, to the polymer dispersions and solutions used in casting or spraying the various layers of the invention delivery composition,. Such pore-forming substances are well known in the art. For example, ammonium bicarbonate salt particles evolve ammonia and carbon dioxide within the solidifying polymer matrix upon solvent evaporation. This method results in a product delivery composition with layers having vacuoles formed therein by gas bubbles. The expansion of pores within the polymer matrix, leads to well interconnected macroporous scaffolds, for example, having mean pore diameters of around 300-400 μm, ideal for high-density in-growth of cells. (Y. S. Nam et al., Journal of Biomedical Materials Research Part B: Applied Biomaterials (2000) 53(1): 1-7). Additional techniques known in the art for creating pores in polymers are the combination of solvent-casting with particulate-leaching, and temperature-induced-phase-separation combined with freeze-drying.

In yet another embodiment, each layer of the delivery composition is cast (e.g., spun by electrospinning) onto the substrate or a preceding layer of the composition as an entanglement of fine polymer fibers, such that a polymer mat or pad is formed upon drying of the layer. Electrospinning produces polymer fibers with diameter in the range of 100 nm and even less, from polymer solutions, suspensions of solid particles and emulsions by spinning a droplet in a field of about 1 kV/cm. The electric force results in an electrically charged jet of polymer solution out-flowing from a droplet tip. After the jet flows away from the droplet in a nearly straight line, the droplet bends into a complex path and other changes in shape occur, during which electrical forces stretch and thin the droplet by very large ratios. After the solvent evaporates, solidified macro to nanofibers are left (D.H. Reneker et al. Nanotechnology (1996) 7:216-223).

The following illustrates estimation of the range of porosity that can be anticipated for the above three exemplary modes of fabrication of a rectangular strip of solid polymer,
wherein SA=surface area of fabrication; V=volume of fabrication; Vp=volume of polymer solid; Nv=total number of pores; R=average radius of pores; and
wherein Vv=total volume of pores is defined by Nv*4/3δR³ Equation 1
   Sv=total surface area of pores is defined by 4δR² Equation 2; and
      P—porosity is defined by VvNp Equation 3
         Porosity of a Standard Rectangular Strip of Solid Film

For a sphere of polymer of radius R, SA/V=3 per unit length, R. For a 1 cm³solid block of polymer, SA/V=6 cm⁻¹, i.e. 6 per unit length. When a 1 cm³ block of solid polymer is pressed into a 1 mm by 1 cm by 10 cm strip, the following holds: SAN=2.04 or ˜2 mm⁻¹ and V=1000 mm³=1 cm³ (as for the cube) So, SA=22.2 cm² and SA/V=˜22.2 cm⁻¹. For each type of fabrication described above, the surface area to volume ratio of the pores (Sv/Vv) will be calculated in terms of porosity (P), so as to

circumvent the necessity of calculating the number of voids, Nv.

Calculations for Equivalent, but Porous Strips or Film

Sv/Vv can be related to SAN most simply when P=1: For P=1, Vv=Vp=V/2. If the practical approximation that Sv>>SA is made, then SA/V=˜2Sv/Vp=k*(Sv/Vp) for constant k=2. When P>1, in each case Sv/Vp increases linearly with P, and SA/V=˜kP*(Sv/Vp)

Film with Drilled Holes (Cylindrical Pores)

For each hole drilled, the surface lost=2*δR² (on two sides) For each hole drilled, the surface gained is=2δR*T. Expressed in terms of porosity, for each hole drilled, the surface lost=2*δR²=2Vv/T, where T is thickness of the film. So, Sv=Nv*[(2δRT)−(2Vv/T)]=(2δRT*Vv/δR^{2*T})−(2Vv/T)=2Vv(1/R−1/T) And, Sv/Vv=2(1/R−1/T) and Sv/Vp=2P(1/R−1/T).

For P=1, Vv=Vp=V/2 and Sv/V=1/R−1/T=˜SA/V. Thus, for 200 μm diameter holes (R=0.01 cm) drilled through the above standard strip (T=0.1 cm, V=1 cm³): SA/V=90 cm⁻¹=4.5*[(SA/V) for solid strip]. Similarly, for holes of half the above diameter (R=0.005 cm): SA/V=190 cm¹=9.5*[(SA/V) for solid strip].

For P>1 the integrity of the essentially two dimensional film, for many applications, may be compromised. However, the theoretical upper limit of porosity can be estimated as follows:

Estimating SA/V is linearly proportional to P (from equations above), for 100 μm holes, and a porosity of 90%: SA/V=9*9.5*[(SA/V) solid strip]=85.5*[(SA/V) for solid strip]; which suggests an upper limit of SA/V˜100x.
For a Sponge (Formed from Gas Bubbles)

Assuming spherical pores of mean radius R: Sv/Vp=Sv*P/Vv=3*P*Nv*4δR**2/Nv*4δR**3=3P/R.

If P=1: Sv/Vp=3/R=˜SA/V. If the mean diameter of pores is 200 um (R=0.01 cm), Sv/Vp=3*10⁵=300 cm⁻¹=˜15*[(SA/V) for a solid strip]. Unlike drilling holes, sponges can be made with very small pores: If mean diameter of pores is 200 nm (R=1*10⁻⁵ cm), Sv/Vp=3*10⁵ cm⁻¹=˜SA/V=15,000*[(SA/V) for solid strip]. Again, Sv/Vp is linearly proportional to porosity, P.

If is P>1: For a porosity of90%, SA/V=9*15,000*[(SA/V) for solid strip]; which suggests an upper porosity limit of P˜150,000x.
For a Fibrous (Electro-Spun) Weave.

The simplest way to approximate an upper limit of SA/V for this mode of fabrication is to model a square grid, in which the linear dimension 2R of the cubic pores is the same as the thickness of the interleaving polymer sheets. Then, Sv/Vp=3/2R. Furthermore, extending this simplest model to increasing Vv/Vp (i.e. increasing porosity, P), yields Sv/Vp=3P/2R.

If P=1: For 200 nm diameter fibers (R=1*10⁻⁵ cm), Sv/Vp=3P//2R=(3*1)/2*1*10⁻⁵ cm or SA/V=7,500*[(SA/V) for solid strip].

If P>1: For a porosity of 90%, SA/V=9*7,500*[(SA/V) for solid strip]; which again suggests an upper limit of P˜70,000x.

In summary then, the broadest envisioned range of surface area over volume ratios for drug-eluting films is between 3 and (150,000×20) cm⁻¹; i.e. 3 to 3,000,000 (3M) cm⁻¹. This range is scaleable; if a very small solid is made with millimeter dimensions, then SA/V can range from 3 to 3M mm⁻¹. Thus, in general, the upper limit of the range will be from 3 to 3M per unit length. That is, the most porous material envisioned could have a surface area to volume ratio of up to one million times the equivalent solid sphere.

However, those of skill in the art would understand that, in practice, the porosity of the invention polymer solid composition should be considered in light of the strength requirements of the particular application for which the composition is intended, with greater porosity being suitable for non-weight bearing applications.

Any solid substrate, such as a stainless steel, or a PolyTetraFluoroEthylene (PTFE) substrate of any shape, preferably planar, such as a disc, can be used for casting or spraying of the various polymer layers that make up the invention solid polymer intraocular delivery compositions. For example in one embodiment, the invention composition is formed as a sandwich of polymer layers, which are formed by pipetting liquid polymer solutions or dispersions onto a stainless steel disc or poly(tetrafluoroethylene) substrate. The substrate can be left in place during manufacture of the invention composition and then removed any time prior to use.

While the bioactive agents can be dispersed within the polymer matrix without chemical linkage to the polymer carrier, it is also contemplated that the bioactive agent or covering molecule can be covalently bound to the biodegradable polymers via a wide variety of suitable functional groups. For example, when the biodegradable polymer is a polyester, the carboxyl group chain end can be used to react with a complimentary moiety on the bioactive agent or covering molecule, such as hydroxy, amino, thio, and the like. A wide variety of suitable reagents and reaction conditions are disclosed, e.g., in March's Advanced Organic Chemistry, Reactions, Mechanisms, and Structure, Fifth Edition, (2001); and Comprehensive Organic Transformations, Second Edition, Larock (1999).

In other embodiments, a bioactive agent can be linked to the PEA, PEUR or PEU polymers described herein through an amide, ester, ether, amino, ketone, thioether, sulfinyl, sulfonyl, disulfide linkage. Such a linkage can be formed from suitably functionalized starting materials using synthetic procedures that are known in the art.

For example, in one embodiment a polymer can be linked to the bioactive agent via an end or pendent carboxyl group (e.g., COOH) of the polymer. For example, a compound of structures IV, VI, and VIII can react with an amino functional group or a hydroxyl functional group of a bioactive agent to provide a biodegradable polymer having the bioactive agent attached via an amide linkage or carboxylic ester linkage, respectively. In another embodiment, the carboxyl group of the polymer can be benzylated or transformed into an acyl halide, acyl anhydride/“mixed” anhydride, or active ester. In other embodiments, the free —NH₂ ends of the polymer molecule can be acylated to assure that the bioactive agent will attach only via a carboxyl group of the polymer and not to the free ends of the polymer.

Water soluble covering molecule(s), such as poly(ethylene glycol) (PEG); phosphoryl choline (PC); glycosaminoglycans including heparin; polysaccharides including polysialic acid; poly(ionizable or polar amino acids) including polyserine, polyglutamic acid, polyaspartic acid, polylysine and polyarginine; chitosan and alginate, as described herein, and targeting molecules, such as antibodies, antigens and ligands, can also be conjugated to the polymer in the exterior of the particles after production of the particles to block active sites not occupied by the bioactive agent or to target delivery of the particles to a specific body site as is known in the art. The molecular weights of PEG molecules on a single particle can be substantially any molecular weight in the range from about 200 to about 200,000, so that the molecular weights of the various PEG molecules attached to the particle can be varied.

Alternatively, the bioactive agent or covering molecule can be attached to the polymer via a linker molecule, for example, as described in structural formulas (XI, XII). Indeed, to improve surface hydrophobicity of the biodegradable polymer, to improve accessibility of the biodegradable polymer towards enzyme activation, and to improve the release profile of the biodegradable polymer, a linker may be utilized to indirectly attach the bioactive agent to the biodegradable polymer. In certain embodiments, the linker compounds include poly(ethylene glycol) having a molecular weight (MW) of about 44 to about 10,000, preferably 44 to 2000; amino acids, such as serine; polypeptides with repeat number from 1 to 100; and any other suitable low molecular weight polymers. The linker typically separates the bioactive agent from the polymer by about 5 angstroms up to about 200 angstroms.

In still further embodiments, the linker is a divalent radical of formula W-A-Q, wherein A is (C₁-C₂₄) alkyl, (C₂-C₂₄) alkenyl, (C₂-C₂₄) alkynyl, (C₃-C₈) cycloalkyl, or (C₆-C₁₀) aryl, and W and Q are each independently —N(R)C(═O)—, —C(═O)N(R)—, —OC(═O)—, —C(═O)O, —O—, —S—, —S(O), —S(O)₂—, —S—S—, —N(R)—, —C(═O)—, wherein each R is independently H or (C₁-C₆) alkyl.

As used to describe the above linkers, the term “alkyl” refers to a straight or branched chain hydrocarbon group including methyl, ethyl, n-propyl, isopropyl, n-butyl, isobutyl, tert-butyl, n-hexyl, and the like.

As used herein to refer to a linker, “alkenyl” refers to straight or branched chain hydrocarbyl groups having one or more carbon-carbon double bonds.

As used herein to refer to a linker, “alkynyl” refers to straight or branched chain hydrocarbyl groups having at least one carbon-carbon triple bond.

As used herein to refer to a linker, “aryl” refers to aromatic groups having in the range of 6 up to 14 carbon atoms.

In certain embodiments, the linker may be a polypeptide having from about 2 up to about 25 amino acids. Suitable peptides contemplated for use include poly-L-glycine, poly-L-lysine, poly-L-glutamic acid, poly-L-aspartic acid, poly-L-histidine, poly-L-omithine, poly-L-serine, poly-L-threonine, poly-L-tyrosine, poly-L-leucine, poly-L-lysine-L-phenylalanine, poly-L-arginine, poly-L-lysine-L-tyrosine, and the like.

In one embodiment, the bioactive agent can covalently crosslink the polymer, i.e. the bioactive agent is bound to more than one polymer molecule. This covalent crosslinking can be done with or without additional polymer-bioactive agent linker.

The bioactive agent molecule can also be incorporated into an intramolecular bridge by covalent attachment between two polymer molecules.

A linear polymer polypeptide conjugate is made by protecting the potential nucleophiles on the polypeptide backbone and leaving only one reactive group to be bound to the polymer or polymer linker construct. Deprotection is performed according to methods well known in the art for deprotection of peptides (Boc and Fmoc chemistry for example).

In one embodiment of the present invention, a polypeptide bioactive agent is presented as retro-inverso or partial retro-inverso peptide.

In other embodiments the bioactive agent is mixed with a photocrosslinkable version of the polymer in a matrix, and after crosslinking the material is dispersed (ground) to an average diameter in the range from about 0.1 to about 10 μm.

The linker can be attached first to the polymer or to the bioactive agent or covering molecule. During synthesis, the linker can be either in unprotected form or protected form, using a variety of protecting groups well known to those skilled in the art. In the case of a protected linker, the unprotected end of the linker can first be attached to the polymer or the bioactive agent or covering molecule. The protecting group can then be de-protected using Pd/H₂ hydrogenolysis, mild acid or base hydrolysis, or any other common de-protection method that is known in the art. The de-protected linker can then be attached to the bioactive agent or covering molecule, or to the polymer

An exemplary synthesis of a biodegradable polymer according to the invention (wherein the molecule to be attached is an aminoxyl) is set forth as follows.

A polyester can be reacted with an amino-substituted aminoxyl (N-oxide) radical bearing group, e.g., 4-amino-2,2,6,6-tetramethylpiperidine-1-oxy, in the presence of N,N′-carbonyldiimidazole to replace the hydroxyl moiety in the carboxyl group at the chain end of the polyester with an amino-substituted aminoxyl-(N-oxide) radical bearing group, so that the amino moiety covalently bonds to the carbon of the carbonyl residue of the carboxyl group to form an amide bond. The N,N′-carbonyl diimidazole or suitable carbodiimide converts the hydroxyl moiety in the carboxyl group at the chain end of the polyester into an intermediate product moiety which will react with the aminoxyl, e.g., 4-amino-2,2,6,6-tetramethylpiperidine-1-oxy. The aminoxyl reactant is typically used in a mole ratio of reactant to polyester ranging from 1: to 100:1. The mole ratio of N,N′-carbonyl diimidazole to aminoxyl is preferably about 1:1.

A typical reaction is as follows. A polyester is dissolved in a reaction solvent and reaction is readily carried out at the temperature utilized for the dissolving. The reaction solvent may be any in which the polyester will dissolve. When the polyester is a polyglycolic acid or a poly(glycolide-L-lactide) (having a monomer mole ratio of glycolic acid to L-lactic acid greater than 50:50), highly refined (99.9+% pure) dimethyl sulfoxide at 115° C. to 130° C. or DMSO at room temperature suitably dissolves the polyester. When the polyester is a poly-L-lactic acid, a poly-DL-lactic acid or a poly(glycolide-L-lactide) (having a monomer mole ratio of glycolic acid to L-lactic acid 50:50 or less than 50:50), tetrahydrofuran, dichloromethane (DCM) and chloroform at room temperature to 40˜50° C. suitably dissolve the polyester.

Polymer—Bioactive agent Linkage

In one embodiment, the polymers used to make the invention polymer particle delivery compositions as described herein have one or more bioactive agent directly linked to the polymer. The residues of the polymer can be linked to the residues of the one or more bioactive agents. For example, one residue of the polymer can be directly linked to one residue of the bioactive agent. The polymer and the bioactive agent can each have one open valence. Alternatively, more than one bioactive agent, multiple bioactive agents, or a mixture of bioactive agents having different therapeutic or palliative activity can be directly linked to the polymer. However, since the residue of each bioactive agent can be linked to a corresponding residue of the polymer, the number of residues of the one or more bioactive agents can correspond to the number of open valences on the residue of the polymer.

As used herein, a “residue of a polymer” refers to a radical of a polymer having one or more open valences. Any synthetically feasible atom, atoms, or functional group of the polymer (e.g., on the polymer backbone or pendant group) of the present invention can be removed to provide the open valence, provided bioactivity is substantially retained when the radical is attached to a residue of a bioactive agent. Additionally, any synthetically feasible functional group (e.g., carboxyl) can be created on the polymer (e.g., on the polymer backbone or pendant group) to provide the open valence, provided bioactivity is substantially retained when the radical is attached to a residue of a bioactive agent. Based on the linkage that is desired, those skilled in the art can select suitably functionalized starting materials that can be derived from the polymer of the present invention using procedures that are known in the art.

As used herein, a “residue of a compound of structural formula (*)” refers to a radical of a compound of polymer formulas (I) and (IV-VIII) as described herein having one or more open valences. Any synthetically feasible atom, atoms, or functional group of the compound (e.g., on the polymer backbone or pendant group) can be removed to provide the open valence, provided bioactivity is substantially retained when the radical is attached to a residue of a bioactive agent. Additionally, any synthetically feasible functional group (e.g., carboxyl) can be created on the compound of formulas (I) and (IV-VIII) (e.g., on the polymer backbone or pendant group) to provide the open valance, provided bioactivity is substantially retained when the radical is attached to a residue of a bioactive agent. Based on the linkage that is desired, those skilled in the art can select suitably functionalized starting materials that can be derived from the compound of formulas (I) and (IV-VIII) using procedures that are known in the art.

For example, the residue of a bioactive agent can be linked to the residue of a compound of structural formula (I) or (IV-VIII) through an amide (e.g., —N(R)C(═O)— or —C(═O)N(R)—), ester (e.g., —OC(═O)— or —C(═O)O—), ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), disulfide (e.g., —S—S—), or a direct (e.g., C-C bond) linkage, wherein each R is independently H or (C₁-C₆) alkyl. Such a linkage can be formed from suitably functionalized starting materials using synthetic procedures that are known in the art. Based on the linkage that is desired, those skilled in the art can select suitably functional starting material that can be derived from a residue of a compound of structural formula (I) or (IV-VIII) and from a given residue of a bioactive agent or adjuvant using procedures that are known in the art. The residue of the bioactive agent or adjuvant can be linked to any synthetically feasible position on the residue of a compound of structural formula (I) or (IV). Additionally, the invention also provides compounds having more than one residue of a bioactive agent or adjuvant bioactive agent directly linked to a compound of structural formula (I) or (IV).

The number of bioactive agents that can be linked to the polymer molecule can typically depend upon the molecular weight of the polymer. For example, for a compound of structural formula (I), wherein n is about 5 to about 150, preferably about 5 to about 70, up to about 150 bioactive agent molecules (i.e., residues thereof) can be directly linked to the polymer (i.e., residue thereof) by reacting the bioactive agent with side groups of the polymer. In unsaturated polymers, the bioactive agents can also be reacted with double (or triple) bonds in the polymer.

The number of bioactive agents that can be linked to the polymer molecule can typically depend upon the molecular weight of the polymer. For example, for a saturated compound of structural formula (I), wherein n is about 5 to about 150, preferably about 5 to about 70, up to about 150 bioactive agents (i.e., residues thereof) can be directly linked to the polymer (i.e., residue thereof) by reacting the bioactive agent with side groups of the polymer. In unsaturated polymers, the bioactive agents can also be reacted with double (or triple) bonds in the polymer.

In one embodiment, the PEA, PEUR and PEU polymers, and mixtures or blends thereof, can be used to formulate biodegradable polymer particle intraocular delivery compositions for time release of at least one ophthalmologic agent in a consistent and reliable manner. These polymer particle delivery compositions can also incorporate an ophthalmologic diol, or a residue of a therapeutic diol or di-acid) into the backbone of the polymer for time release of the bioactive agent from the backbone of the polymer in a consistent and reliable manner by biodegradation of the polymers in the polymer particles.

PEA, PEUR and PEU polymers described herein absorb water, (5 to 25% w/w water up-take, on polymer film) allowing hydrophilic molecules to readily diffuse therethrough. This characteristic makes these polymers suitable for use as an over coating on particles to control release rate. Water absorption also enhances biocompatibility of the polymers and the polymer particle delivery composition based on such polymers. In addition, due to the hydrophilic properties of the PEA, PEUR and PEU polymers, when delivered intraocularly the particles become sticky and agglomerate, particularly at in vivo temperatures. Thus the polymer particles spontaneously form polymer depots when injected intraocularly for local delivery. Particles with average diameter range from about I micron to about 100 microns, which size will not circulate efficiently within the body, are suitable for forming such polymer depots intraocularly.

Methods of making intraocular polymer particle delivery compositions Particles suitable for use in the invention intraocular polymer delivery composition can be made using immiscible solvent techniques and as described herein and in copending U.S. applications Nos. 60/684,670, filed May 25, 2005; 60/737,401, filed Nov. 14, 2005; 60/687,570, filed Jun. 3, 2005; 60/759,179, filed Jan. 13, 2006 (Atty Docket MEDIV2070-1); 60/719,950, filed Sep. 22, 2005, each of which is incorporated herein by reference in its entirety. Generally, these methods entail the preparation of an emulsion of two immiscible liquids. A single emulsion method can be used to make polymer particles that incorporate at least one hydrophobic bioactive agent. In the single emulsion method, bioactive agents to be incorporated into the particles are mixed with polymer in solvent first, and then emulsified in water solution with a surface stabilizer, such as a surfactant. In this way, polymer particles with hydrophobic bioactive agent conjugates are formed and suspended in the water solution, in which hydrophobic conjugates in the particles will be stable without significant elution into the aqueous solution, but such molecules will elute into body tissue, such as muscle tissue.

Most biologics, including polypeptides, proteins, DNA, cells and the like, are hydrophilic. A double emulsion method can be used to make polymer particles with interior aqueous phase and hydrophilic bioactive agents dispersed within. In the double emulsion method, aqueous phase or hydrophilic bioactive agents dissolved in water are emulsified in polymer lipophilic solution first to form a primary emulsion, and then the primary emulsion is put into water to emulsify again to form a second emulsion, in which particles are formed having a continuous polymer phase and aqueous bioactive agent(s) in the dispersed phase. Surfactant and additive can be used in both emulsifications to prevent particle aggregation. Chloroform or DCM, which are not miscible in water, are used as solvents for PEA and PEUR polymers, but later in the preparation the solvent is removed, using methods known in the art.

For certain bioactive agents with low water solubility, however, these two emulsion methods have limitations. In this context, “low water solubility” means a bioactive agent that is less hydrophobic than truly lipophilic drugs, such as Taxol, but which are less hydrophilic than truly water-soluble drugs, such as many biologics. These types of intermediate compounds are too hydrophilic for high loading and stable matrixing into single emulsion particles, yet are too hydrophobic for high loading and stability within double emulsions. In such cases, a polymer layer is coated onto particles made of polymer and drugs with low water solubility, by a triple emulsion process. This method provides relatively low drug loading (˜10% w/w), but provides structure stability and controlled drug release rate.

In the triple emulsion process, the first emulsion is made by mixing the bioactive agents into polymer solution and then emulsifying the mixture in aqueous solution with surfactant or lipid, such as di-(hexadecanoyl) phosphatidylcholine (DHPC; a short-chain derivative of a natural lipid). In this way, particles containing the active agents are formed and suspended in water to form the first emulsion. The second emulsion is formed by putting the first emulsion into a polymer solution, and emulsifying the mixture, so that water drops with the polymer/drug particles inside are formed within the polymer solution. Water and surfactant or lipid will separate the particles and dissolve the particles in the polymer solution. The third emulsion is then formed by putting the second emulsion into water with surfactant or lipid, and emulsifying the mixture to form the final particles in water. The resulting particle structure will have one or more particles made with polymer plus bioactive agent at the center, surrounded by water and surface stabilizer, such as surfactant or lipid, and covered with a pure polymer shell. Surface stabilizer and water will prevent solvent in the polymer coating from contacting the particles inside the coating and dissolving them.

To increase loading of bioactive agents by the triple emulsion method, active agents with low water solubility can be coated with surface stabilizer in the first emulsion, without polymer coating and without dissolving the bioactive agent in water. In this first emulsion, water, surface stabilizer and active agent have similar volume or in the volume ratio range of (1 to 3):(0.2 to about 2): 1, respectively. In this case, water is used, not for dissolving the active agent, but rather for protecting the bioactive agent with help of surface stabilizer. Then the double and triple emulsions are prepared as described above. This method can provide up to 50% drug loading.

Alternatively or additionally in the single, double or triple emulsion methods described above, a bioactive agent can be conjugated to the polymer molecule as described herein prior to using the polymers to make the particles. Alternatively still, a bioactive agent can be conjugated to the polymer on the exterior of the particles described herein after production of the particles.

Many emulsification techniques will work in making the emulsions described above. However, the presently preferred method of making the emulsion is by using a solvent that is not miscible in water. For example, in the single emulsion method, the emulsifying procedure consists of dissolving polymer with the solvent, mixing with bioactive agent molecule(s), putting into water, and then stirring with a mixer and/or ultra-sonicator. Particle size can be controlled by controlling stir speed and/or the concentration of polymer, bioactive agent(s), and surface stabilizer. Coating thickness can be controlled by adjusting the ratio of the second to the third emulsion.

Suitable emulsion stabilizers may include nonionic surface active agents, such as mannide monooleate, dextran 70,000, polyoxyethylene ethers, polyglycol ethers, and the like, all readily commercially available from, e.g., Sigma Chemical Co., St. Louis, Mo. The surface active agent will be present at a concentration of about 0.3% to about 10%, preferably about 0.5% to about 8%, and more preferably about 1% to about 5%.

Rate of release of the at least one bioactive agent from the invention compositions can be controlled by adjusting the coating thickness, particle size, structure, and density of the coating. Density of the coating can be adjusted by adjusting loading of the bioactive agent conjugated to the coating. For example, when the coating contains no bioactive agent, the polymer coating is densest, and a bioactive agent from the interior of the particle elutes through the coating most slowly. By contrast, when a bioactive agent is loaded into (i.e. is mixed or “matrixed” with), or alternatively is conjugated to, polymer in the coating, the coating becomes porous once the bioactive agent has become free of polymer and has eluted out, starting from the outer surface of the coating. Thereby, a bioactive agent at the center of the particle can elute at an increased rate. The higher the bioactive agent loading in the coating, the lower the density of the coating layer and the higher the elution rate. The loading of bioactive agent in the coating can be lower or higher than that in the interior of the particles beneath the exterior coating. Release rate of bioactive agent(s) from the particles can also be controlled by mixing particles with different release rates prepared as described above.

A detailed description of methods of making double and triple emulsion polymers may be found in Pierre Autant et al, Medicinal and/or nutritional microcapsules for oral administration, U.S. Pat. No. 6,022,562; Iosif Daniel Rosca et al., Microparticle formation and its mechanism in single and double emulsion solvent evaporation, Journal of Controlled Release 99 (2004) 271-280; L. Mu, S. S. Feng, A novel controlled release formulation for the anticancer drug paclitaxel (Taxol): PLGA nanoparticles containing vitamin E TPGS, J. Control. Release 86 (2003) 33-48; Somatosin containing biodegradable microspheres prepared by a modified solvent evaporation method based on W/O/W-multiple emulsions, Int. J. Pharm. 126 (1995) 129- 138 and F. Gabor, B. Ertl, M. Wirth, R. Mallinger, Ketoprofenpoly(d,l-lactic-co-glycolic acid) microspheres: influence of manufacturing parameters and type of polymer on the release characteristics, J. Microencapsul. 16 (1) (1999) 1-12, each of which is incorporated herein in its entirety.

In yet further embodiments for delivery of aqueous-soluble bioactive agents, the particles can be made into nanoparticles having an average diameter of about 20 nm to about 200 nm for delivery to the circulation. The nanoparticles can be made by the single emulsion method with the active agent dispersed therein, i.e., mixed into the emulsion or conjugated to polymer as described herein. The nanoparticles can also be provided as a micellar composition containing the polymers described herein, such as PEA and PEUR with the bioactive agents conjugated thereto. Alternatively or in addition to bioactive agents conjugated to the polymers, since the micelles are formed in water, water soluble bioactive agents can be loaded into the micelles at the same time without solvent.

More particularly, the biodegradable micelles are formed of a hydrophobic polymer chain conjugated to a water soluble polymer chain. Whereas, the outer portion of the micelle mainly consists of the water soluble ionized or polar section of the polymer, the hydrophobic section of the polymer mainly partitions to the interior of the micelles and holds the polymer molecules together.

The biodegradable hydrophobic section of the polymer used to make micelles is made of PEA, PEUR or PEU polymers, as described herein. For strongly hydrophobic PEA, PEUR or PEU polymers, components such as di- L-leucine ester of 1,4:3,6-dianhydro-D-sorbitol or rigid aromatic di-acid like α,ω-bis (4-carboxyphenoxy) (C₁-C₈) alkane may be included in the polymer repeat unit. By contrast, the water soluble section of the polymer comprises repeating alternating units of polyethylene glycol, polyglycosaminoglycan or polysaccharide and at least one ionizable or polar amino acid, wherein the repeating alternating units have substantially similar molecular weights and wherein the molecular weight of the polymer is in the range from about 10 kDa to about 300 kDa. The repeating alternating units may have substantially similar molecular weights in the range from about 300 Da to about 700 Da. In one embodiment wherein the molecular weight of the polymer is over 10 kDa, at least one of the amino acid units is an ionizable or polar amino acid selected from serine, glutamic acid, aspartic acid, lysine and arginine. In one embodiment, the units of ionizable amino acids comprise at least one block of ionizable poly(amino acids), such as glutamate or aspartate, can be included in the polymer. The invention micellar composition may further comprise a pharmaceutically acceptable aqueous media with a pH value at which at least a portion of the ionizable amino acids in the water soluble sections of the polymer are ionized.

The higher the molecular weight of the water soluble section of the polymer, the greater the porosity of the micelle and the higher the loading into the micelles of water soluble bioactive agents and/or large bioactive agents, such as proteins. In one embodiment, therefore, the molecular weight of the complete water soluble section of the polymer is in the range from about 5 kDa to about 100 kDa.

Once formed, the micelles can be lyophilized for storage and shipping and reconstituted in the above-described aqueous media. However, it is not recommended to lyophilize micelles containing certain bioactive agents, such as certain proteins, that would be denatured by the lyophilization process.

Charged moieties within the micelles partially separate from each other in water, and create space for absorption of water soluble agents, such as the bioactive agent(s). Ionized chains with the same type of charge will repel each other and create more space. The ionized polymer also attracts the bioactive agent, providing stability to the matrix. In addition, the water soluble exterior of the micelle prevents adhesion of the micelles to proteins in body fluids after ionized sites are taken by the therapeutic bioactive agent. This type of micelle has very high porosity, up to 95% of the micelle volume, allowing for high loading of aqueous-soluble biologics, such as polypeptides, DNA, and other bioactive agents. Particle size range of the micelles is about 20 nm to about 200 nm, with about 20 nm to about 100 nm being preferred for circulation in the blood.

Particle size can be determined by, e.g., laser light scattering, using for example, a spectrometer incorporating a helium-neon laser. Generally, particle size is determined at room temperature and involves multiple analyses of the sample in question (e.g., 5-10 times) to yield an average value for the particle diameter. Particle size is also readily determined using scanning electron microscopy (SEM). In order to do so, dry particles are sputter-coated with a gold/palladium mixture to a thickness of approximately 100 Angstroms, and then examined using a scanning electron microscope. Alternatively, the polymer, either in the form of particles or not, can be covalently attached directly to the bioactive agent, rather than incorporating active agent therein (“loading) without chemical attachment, using any of several methods well known in the art and as described hereinbelow. The bioactive agent content is generally in an amount that represents approximately 0.1% to about 40% (w/w) bioactive agent to polymer, more preferably about 1% to about 25% (w/w) bioactive agent, and even more preferably about 2% to about 20% (w/w) bioactive agent. The percentage of bioactive agent will depend on the desired dose and the condition being treated, as discussed in more detail below.

Methods of making intraocular solid polymer delivery compositions To fabricate the invention intraocular solid polymer delivery composition for controlled release of an ophthalmologic agent, the following polymer layers can be cast or sprayed onto a solid substrate:
a) at least one carrier layer comprising a liquid solution in a first solvent of at least one bioactive agent and a biodegradable, biocompatible polymer having a structural formula described by structural formula (I)-(IV-VIII) as described herein;
b) at least one coating layer of a liquid solution in a second solvent of a biodegradable, biocompatible polymer; and
c) at least one barrier layer of a liquid polymer that is insoluble in the second solvent, but dissolves under physiological conditions, wherein the barrier layer is between the carrier layer and each coating layer. Each layer is dried before casting or spraying the next layer thereon. A polymer layer cast or sprayed onto the substrate as a liquid dispersion forms a film (for example, a substantially planar body having opposed major surfaces and a thickness between the major surfaces of from about 0.1 millimeters to about 20 millimeters, e.g. 5 millimeters).

Alternatively, for constructs having a thickness of about 0.1 to 2.5 mm in thickness, a carrier layer comprising a liquid solution in a first solvent of at least one biodegradable, biocompatible polymer with dispersed ophthalmologic agent is sprayed onto the solid substrate and dried. Then a single coating layer of a liquid solution of a biodegradable, biocompatible polymer in the same solvent or in a second solvent is sprayed atop the carrier layer and dried.

Drying of the various layers can be accomplished using any method known in the art so long as the temperature is not high enough to breakdown the chemical structure of the various polymers used or that of the one or more bioactive agents dispersed in the carrier layer. Typically, temperatures do not exceed 80° C. For example, the various layers of the composition can be dried in an oven at a temperature of 40° C. to about 50° C. for a period of about 5 hours to about 9 hours, for example, about 7 hours at 50° C., as is illustrated in the Examples herein.

In one embodiment, the coating layer(s) as well as the carrier layer are applied using a liquid solution of a polymer having a chemical formula described by structural formulas (I) and (IV). Although in no way necessary for practice of the invention, for convenience, it is recommended to use the same combination of solvent and biodegradable, biocompatible polymer of structures (I)-(IV) in the coating layer(s) as in the carrier layer(s) of the invention composition. For example, the polymer used in casting or spraying the coating layer(s) can be the same as that used in casting or spraying the carrier layer(s), in which case the first solvent and the second solvent can be identical.

The composition of the barrier layer(s) in the invention solid polymer intraocular delivery composition is an important aspect of the invention. The choice of the polymer used in the barrier layer(s) is determined by the solvent used to form the solution for preparation of the covering layer(s). The purpose of the barrier layer(s) being to prevent uncontrolled solvation of the bioactive agent out of the carrier layer(s) during deposition of the covering layer with which it would otherwise be in contact. The polymer for the intermediate barrier layer(s) is selected to be insoluble in solvent used in formation of the covering layer(s). In one embodiment, the barrier layer is a monolayer of polymer in the finished composition. In addition, all polymers used in the various layers of the solid polymer delivery composition are biocompatible and will be re-absorbed by the body through natural enzymatic action. In another embodiment, the coating layer(s) applied are free of bioactive agents and may be referred to herein as a “pure polymer layer”.

For example, a polymer that dissolves in ethanol, such as copolymer PEA (a form of the polymer represented in formula (IV)), can be used in a solution of this solvent in applying the carrier and coating layers, and a polyvinyl alcohol, which is insoluble in ethanol, can be used to make one or more barrier layers in the composition. In one embodiment, the liquid polymer that forms the barrier layer(s) is applied so as to form a monolayer of the polymer, e.g., polyvinyl alcohol.

To further prolong the release period from the invention solid polymer intraocular delivery composition, the composition has a three-dimensional sandwich structure. The method of making the composition may then be modified by first casting multiple sets of the coating layer and barrier layer onto the substrate, beginning with a coating layer (drying each before deposit of the next) prior to casting the carrier layer. Then, to form the interior of the sandwich structure, the carrier layer is cast and dried. Finally, in reverse order, multiple sets of the barrier layer and coating layer are cast atop the dried barrier layer. In this way, a coating layer is first and last to be deposited and the composition takes on a sandwich structure, wherein the multiple sets are applied twice to form two external sides of a three-dimensional sandwich structure, with a coating layer being external to both of the two sides of the structure and with the carrier layer being at the center of the sandwich structure. Each composition construct, therefore, may contain no set of the layers or about one to about ten barrier and coating layer sets (e.g., 1 to about 8 sets of barrier and coating layers).

In a more schematic format, for this embodiment the following procedure can be followed to make a composition having N sets of coating and barrier layers in a sandwich structure, with the carrier layer at the center of the sandwich structure:
1. Cast/spray Nth layer of coating onto substrate and dry,
2. Coat with Nth barrier layer and dry,
3. Cast/spray (N−1)th coating layer and dry,
4. Coat with (N−1)th barrier layer and dry,
5. Repeat steps 3 and 4 as desired.
6. Cast/spray carrier layer containing one or more matrixed bioactive agent (the carrier layer will end up as the inner-most layer of the sandwich) and dry.
7. Repeat steps 5 to 1 in the reverse order, ending with a coating layer. Preferably each liquid layer deposited will over run the edge of the previous one to seal the sides of the composition being formed so that elution from the sides of the disc will be controlled as well as from other portions of the surface area. The substrate can be removed from the dried layers at any point in the method of making the invention composition after one or two layers have been cast and dried thereon. The completed composition can be packaged for storage or is ready for immediate use.

Alternatively, if the solvent is not completely removed from the various layers of the composition during the drying steps described above, the composition can be manipulated and compressed to form any of a number of three dimensional shapes, such as by rolling, pleating, folding, and the like, prior to a final drying to substantially remove solvent from the composition. For example, a disc can be rolled up and compressed to form a cylinder. Alternatively, cylinders can be punched with a dye from a sheet of fully dried material formed layer by layer according to the above described methods.

For thinner composition constructs, primarily where the total thickness of the construct does not exceed approximately 2.5 mm, an aerosol of solvated polymer matrixed with bioactive molecule(s) is deposited on a substrate. Alternatively, the carrier layer can be solution cast. This carrier layer is then dried by methodology outlined in c). For the deposition of the coating layer(s) using an aerosol, diffusion of the drug or biologic out of the carrier layer and into the wet coating layer being formed is limited by three factors: 1) concentration of the biodegradable, biocompatible polymer solution is typically as high as possible (c.a. 2% to 3%) without forming solids in the air, such that the aerosol is nearly dry upon deposition on the surface, 2) substrate and/or airspace above are continuously heated to promote rapid drying (typically 30 to 80° C., depending on the polymer and concentration), and 3) a single coat is divided into many “spray cycles.” By using “spray cycles,” the period of deposition of the aerosol is divided into many shorter periods of less than a second in length to several seconds. Each spray period is followed by a short drying period (e.g. 20 to 60 seconds, incorporating continuous heating in each cycle), such that drying is rapid and residual solvent is minimized for a given coating. Heat can typically be applied continuously, even during the aerosol deposition periods of the spray cycle. Drying of the newly formed coating layer is performed as described herein. Any single coat typically does not exceed approximately 80 μm. Although migration of the biologic and/or drug into the newly formed coating layer may not be completely eliminated as in embodiments employing the barrier layer, migration is limited to the extent that diffusion of small molecules in the final product is slower than in those embodiments containing only a carrier layer. The rate of release of the ophthalmologic agent and optional bioactive molecules is thereby controlled.

Methods of making solid polymer implantables containing PEA, PEUR and PEU polymers are further disclosed in U.S. application Ser. No. 11/525,491, filed Sep. 21, 2006, which is incorporated herein by reference in its entirety.

Bioactive agents for optional dispersion into and release from the invention biodegradable intraocular polymer delivery compositions also include anti-proliferants, rapamycin and any of its analogs or derivatives, paclitaxel or any of its taxene analogs or derivatives, everolimus, Sirolimus, tacrolimus, or any of its—limus named family of drugs, and statins such as simvastatin, atorvastatin, fluvastatin, pravastatin, lovastatin, rosuvastatin, geldanamycins, such as 17AAG (17-allylamino-17-demethoxygeldanamycin); Epothilone D and other epothilones, 17-dimethylaminoethylamino-17-demethoxy-geldanamycin and other polyketide inhibitors of heat shock protein 90 (Hsp90), Cilostazol, and the like.

The following bioactive agents and small molecule drugs can optionally be dispersed within the invention intraocular delivery compositions, whether formulated and sized to form a time release biodegradable polymer depot for local delivery of the bioactive agents or fabricated as solid delivery systems. Any optional bioactive agents that are dispersed in the polymers used in the invention delivery compositions and methods of delivery will be selected for their suitable therapeutic or palliative effect in treatment of an ophthalmologic disease of interest, or symptoms thereof.

In one embodiment, the optional bioactive agents are not limited to, but include, various classes of compounds that facilitate or contribute to wound healing when presented in a time-release fashion.

Small molecule drugs are a category of additional bioactive agents suitable for dispersion in the invention intraocular polymer delivery compositions described herein. Such drugs include, for example, antimicrobials and anti-inflammatory agents as well as certain healing promoters, such as, for example, vitamin A and synthetic inhibitors of lipid peroxidation.

A variety of antibiotics can be dispersed in the invention intraocular polymer delivery compositions to indirectly promote natural healing processes by preventing or controlling infection. Suitable antibiotics include many classes, such as aminoglycoside antibiotics or quinolones or beta-lactams, such as cefalosporins, e.g., ciprofloxacin, gentamycin, tobramycin, erythromycin, vancomycin, oxacillin, cloxacillin, methicillin, lincomycin, ampicillin, and colistin. Suitable antibiotics have been described in the literature.

Suitable antimicrobials include, for example, Adriamycin PFS/RDF® (Pharmacia and Upjohn), Blenoxane® (Bristol-Myers Squibb Oncology/Immunology), Cerubidine® (Bedford), Cosmegen® (Merck), DaunoXome® (NeXstar), Doxil® (Sequus), Doxorubicin Hydrochloride® (Astra), Idamycin® PFS (Pharmacia and Upjohn), Mithracin® (Bayer), Mitamycin® (Bristol-Myers Squibb Oncology/Immunology), Nipene (SuperGen), Novantrone® (Immunex) and Rubex® (Bristol-Myers Squibb Oncology/Immunology). In one embodiment, the peptide can be a glycopeptide. “Glycopeptide” refers to oligopeptide (e.g. heptapeptide) antibiotics, characterized by a multi-ring peptide core optionally substituted with saccharide groups, such as vancomycin.

Examples of glycopeptides included in this category of antimicrobials may be found in “Glycopeptides Classification, Occurrence, and Discovery,” by Raymond C. Rao and Louise W. Crandall, (“Bioactive agents and the Pharmaceutical Sciences” Volume 63, edited by Ramakrishnan Nagarajan, published by Marcal Dekker, Inc.). Additional examples of glycopeptides are disclosed in U.S. Pat. Nos. 4,639,433; 4,643,987; 4,497,802; 4,698,327, 5,591,714; 5,840,684; and 5,843,889; in EP 0 802 199;.EP 0 801 075; EP 0 667 353; WO 97/28812; WO 97/38702; WO 98/52589; WO 98/52592; and in J. Amer. Chem. Soc., 1996, 118, 13107-13108; J. Amer. Chem. Soc., 1997, 119, 12041-12047; and J. Amer. Chem. Soc., 1994, 116, 4573-4590. Representative glycopeptides include those identified as A477, A35512, A40926, A41030, A42867, A47934, A80407, A82846, A83850, A84575, AB-65, Actaplanin, Actinoidin, Ardacin, Avoparcin, Azureomycin, Balhimyein, Chloroorientiein, Chloropolysporin, Decaplanin, -demethylvancomycin, Eremomycin, Galacardin, Helvecardin, Izupeptin, Kibdelin, LL-AM374, Mannopeptin, MM45289, MM47756, MM47761, MM49721, MM47766, MM55260, MM55266, MM55270, MM56597, MM56598, OA-7653, Orenticin, Parvodicin, Ristocetin, Ristomycin, Synmonicin, Teicoplanin, UK-68597, UD-69542, UK-72051, Vancomycin, and the like. The term “glycopeptide” or “glycopeptide antibiotic” as used herein is also intended to include the general class of glycopeptides disclosed above on which the sugar moiety is absent, i.e. the aglycone series of glycopeptides. For example, removal of the disaccharide moiety appended to the phenol on vancomycin by mild hydrolysis gives vancomycin aglycone. Also included within the scope of the term “glycopeptide antibiotics” are synthetic derivatives of the general class of glycopeptides disclosed above, included alkylated and acylated derivatives. Additionally, within the scope of this term are glycopeptides that have been further appended with additional saccharide residues, especially aminoglycosides, in a manner similar to vancosamine.

The term “lipidated glycopeptide” refers specifically to those glycopeptide antibiotics that have been synthetically modified to contain a lipid substituent. As used herein, the term “lipid substituent” refers to any substituent contains 5 or more carbon atoms, preferably, 10 to 40 carbon atoms. The lipid substituent may optionally contain from 1 to 6 heteroatoms selected from halo, oxygen, nitrogen, sulfur, and phosphorous. Lipidated glycopeptide antibiotics are well known in the art. See, for example, in U.S. Pat. Nos. 5,840,684, 5,843,889, 5,916,873, 5,919,756, 5,952,310, 5,977,062, 5,977,063, EP 667, 353, WO 98/52589, WO 99/56760, WO 00/04044, WO 00/39156, the disclosures of which are incorporated herein by reference in their entirety.

Anti-inflammatory bioactive agents are also useful for dispersion in polymer particles used in invention compositions and methods. Depending on the intraocular site and disease to be treated, such anti-inflammatory bioactive agents include, e.g. analgesics (e.g., NSAIDS and salicyclates), steroids, antirheumatic agents, gastrointestinal agents, gout preparations, hormones (glucocorticoids), nasal preparations, ophthalmic preparations, otic preparations (e.g., antibiotic and steroid combinations), respiratory agents, and skin & mucous membrane agents. See, Physician's Desk Reference, 2001 Edition. Specifically, the anti-inflammatory agent can include dexamethasone, which is chemically designated as (11δ, 16I)-9-fluro-11,17,21-trihydroxy-16-methylpregna-1,4-diene-3,20-dione. Alternatively, the anti-inflammatory bioactive agent can be or include sirolimus (rapamycin), which is a triene macrolide antibiotic isolated from Streptomyces hygroscopicus.

The polypeptide bioactive agents included in the invention compositions and methods can also include “peptide mimetics.” Such peptide analogs, referred to herein as “peptide mimetics” or “peptidomimetics,” are commonly used in the pharmaceutical industry with properties analogous to those of the template peptide (Fauchere, J. (1986) Adv. Bioactive agent Res., 15: 29; Veber and Freidinger (1985) TINS p. 392; and Evans et al. (1987) J. Med. Chem., 30: 1229) and are usually developed with the aid of computerized molecular modeling. Generally, peptidomimetics are structurally similar to a paradigm polypeptide (i.e., a polypeptide that has a biochemical property or pharmacological activity), but have one or more peptide linkages optionally replaced by a linkage selected from the group consisting of: —CH₂NH—, —CH₂S—, CH₂-CH₂—, —CH═CH— (cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends. Pharm. Sci., (1 980) pp. 463-468 (general review); Hudson, D. et al., Int. J. Pept. Prot. Res., (1979) 14: 177-185 (—CH₂NH—, CH₂CH₂—); Spatola, A. F. et al., Life Sci., (1986) 38: 1243-1249 (—CH₂—S—); Harm, M. M., J. Chem. Soc. Perkin Trans I (1982) 307-314 (—CH═CH—, cis and trans); Almquist, R. G. et al., J. Med. Chem., (1980) 23: 2533 (—COCH₂—); Jennings-Whie, C. et al., Tetrahedron Lett., (1982) 23: 2533 (—COCH₂—); Szelke, M. et al., European Appln., EP 45665 (1982) CA: 97: 39405 (1982) (—CH(OH)CH₂—-); Holladay, M. W. et al., Tetrahedron Lett., (1983) 24: 4401-4404 (—C(OH)CH₂—); and Hruby, V. J., Life Sci., (1982) 31: 189-199 (--CH₂-S--). Such peptide mimetics may have significant advantages over natural polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Additionally, substitution of one or more amino acids within a peptide (e.g., with a D-Lysine in place of L-Lysine) may be used to generate more stable peptides and peptides resistant to endogenous peptidases. Alternatively, the synthetic polypeptides covalently bound to the biodegradable polymer, can also be prepared from D-amino acids, referred to as inverso peptides. When a peptide is assembled in the opposite direction of the native peptide sequence, it is referred to as a retro peptide. In general, polypeptides prepared from D-amino acids are very stable to enzymatic hydrolysis. Many cases have been reported of preserved biological activities for retro-inverso or partial retro-inverso polypeptides (U.S. Pat. No. 6,261,569 B1 and references therein ; B. Fromme et al, Endocrinology (2003)144: 3262-3269.

Following preparation of the polymer particle compositions loaded with ophthalmologic agent, the composition can be lyophilized and the dried composition suspended in an appropriate media prior to administration.

Any suitable and effective amount of the at least one ophthalmologic agent can be released with time from the polymer particles (including those in a polymer depot formed in vivo) or solid polymer formulation and will typically depend, e.g., on the specific polymer, type of particle or polymer/bioactive agent linkage, if present. Typically, up to about 100% of the polymer particles can be released from a polymer depot formed in vivo by particles sized to avoid circulation. Specifically, up to about 90%, up to 75%, up to 50%, or up to 25% thereof can be released from the polymer depot. Factors that typically affect the release rate from the polymer are the nature and amount of the polymer/ophthalmologic agent, the types of polymer/ophthalmologic agent linkage, and the nature and amount of additional substances present in the formulation.

Once the invention polymer particle delivery composition is made, as above, compositions are formulated for subsequent intraocular. The particle-containing compositions will generally include one or more “pharmaceutically acceptable excipients or vehicles” appropriate for implant into the interior of the eye, such as water, saline, glycerol, polyethylene glycol, hyaluronic acid, ethanol, etc. Additionally, auxiliary substances, such as wetting agents, pH buffering substances, and the like, may be present in such vehicles.

For a further discussion of appropriate vehicles to use for particular modes of delivery, see, e.g., Remington: The Science and Practice of Pharmacy, Mack Publishing Company, Easton, Pa., 19th edition, 1995. One of skill in the art can readily determine the proper vehicle to use for the particular bioactive agent/polymer particle combination, size of particle and mode of administration.

The compositions used in the invention methods optionally may comprise an “effective amount” of the ophthalmologic agent(s) of interest, such as an ophthalmologic diol incorporated into the backbone of the PEA, PEUR or PEU polymer. That is, an amount of an ophthalmologic agent may be included in the compositions that will cause the subject to produce a sufficient therapeutic or palliative response in order to prevent, reduce or eliminate symptoms. The exact amount necessary will vary, depending on the subject being treated; the age and general condition of the subject to be treated; the severity of the condition being treated; the particular ophthalmologic agent selected and formulation, among other factors. An appropriate effective amount can be readily determined by one of skill in the art. Thus, an “effective amount” will fall in a relatively broad range that can be determined through routine trials. For example, for purposes of the present invention, an effective amount will typically range from about 1 μg to about 100 mg, for example from about 5 μg to about 1 mg, or about 10 μg to about 500 μg of the active agent delivered per dose.

Once formulated, the invention polymer article delivery compositions can be administered by implant into the interior of the eye, for example by injection through a trochar or needle or by insertion via a surgical incision. Dosage treatment may be a single dose of the invention polymer particle delivery composition, or a multiple dose schedule as is known in the art. The dosage regimen, at least in part, will also be determined by the need of the subject and be dependent on the judgment of the practitioner. Furthermore, if prevention of disease is desired, the polymer particle delivery composition is generally administered prior to primary disease manifestation, or symptoms of the disease of interest. If treatment is desired, e.g., the reduction of symptoms or recurrences, the polymer particle delivery compositions are generally administered subsequent to primary disease manifestation.

The following examples are meant to illustrate, but not to limit the invention.

### EXAMPLE 1

Preparation of PEA-Ac-Bz Nanoparticles and Particles by the Single Emulsion Method

PEA polymer of structure (IV) containing acetylated ends and benzylated carboxyl pendent groups COOCH₂C₆H₅ (designated as PEA.Ac.Bz) (25 mg) was dissolved in 1 ml of DCM and added to 5 ml of 0.1% surfactant diheptanoyl-phosphatidylcholine (DHPC) in aqueous solution while stirring. After rotary-evaporation, a PEA.Ac.Bz emulsion with particle sizes ranging from 20 nm to 100 μm, was obtained. The higher the stir rate, the smaller the sizes of particles. Particle size is controlled by molecular weight of the polymer, solution concentration and equipment such as microfluidizer, ultrasound sprayer, sonicator, and mechanical or magnetic stirrer.

Preparation of PEA.Ac.Bz Particles Containing a Pain Killer

PEA.Ac.Bz (25 mg) and Bupivicane (5 mg) were dissolved in 1 mL of DCM and the solution was added to 5 mL of 0.1% DHPC aqueous solution while homogenizing. Using a rotary evaporator, a PEA.Ac.Bz emulsion with average particle size ranging from 0.5 μm to 1000 μm, preferentially, from 1 μm to about 20 μm, have been made.

### EXAMPLE 2

Preparation of Polymer Particles Using a Double Emulsion Method

Particles were prepared using a double emulsion technique in two steps: in the first step, PEA.Ac.Bz (25 mg) was dissolved in 1 mL of DCM, and then 50 μL of 10% surfactant diheptanoyl-phosphatidylcholine (DHPC), was added. The mixture was vortexed at room temperature to form a Water/Oil (W/O) primary emulsion. In the second step, the primary emulsion was added slowly into a 5 mL solution of 0.5% DHPC while homogenizing the mixed solution. After 1 min of homogenization, the emulsion was rotary-evaporated to remove DCM to obtain a Water/Oil/Water double emulsion. The generated double emulsion had suspended polymer particles with sizes ranging from 0.5 μm to 1000 μm, with most about 1 μm to 10 μm . Reducing such factors as the amount of surfactant, the stir speed and the volume of water, tends to increase the size of the particles.

### EXAMPLE 3

Preparation of PEA Particles Encapsulating an Antibody Using a Double Emulsion Method

Particles were prepared using the double emulsion technique by two steps: in the first step, PEA.Ac.Bz (25 mg) was dissolved in I mL of DCM, and then 50 μL of aqueous solution containing 60 μg of anti-Icam-1 antibody and 4.0 mg of DHPC were added. The mixture was vortexed at room temperature to form a Water/Oil primary emulsion. In the second step, the primary emulsion was added slowly into 5 mL of 0.5% DHPC solution while homogenizing. After 1 min of homogenization, the emulsion was rotary-evaporated to remove DCM to obtain particles having a Water/Oil/Water (W/O/W) double emulsion structure. About 75% to 98% of antibody was encapsulated by using this double emulsion technique.

### EXAMPLE 4

Preparation of Particles having a Triple Emulsion Structure, Wherein One or More Primary Particles are Encapsulated Together Within a Polymer Covering to Form Secondary Microparticles.

Particles having a triple emulsion structure have been prepared by the following two different routes:

Multi-particle Encapsulation By the first route, primary particles were prepared using a standard procedure for single phase, PEA.Ac.H (polymer of structure (IV) containing acetylated ends and free COOH pendent groups) nanoparticles were prepared to afford a stock sample, ranging from about 1.0 mg to about 10 mg/mL (polymer per aqueous unit). In addition, a solution of the PEA.Ac.Bz stock sample, with a 20% surfactant weight amount wherein the 20% is calculated as (milligrams of surfactant)/(milligrams of PEA.Ac.Bz+milligrams of surfactant) was prepared. Various surfactants were explored, with the most successful being 1,2-Diheptanoyl-sn-glycero-3-phosphocholine. The stock sample of PEA.Ac.H nanoparticles was injected into a solution of PEA.Ac.Bz polymer in DCM. A typical example was as follows:

| | |
|---|---|
| Nanoparticle Stock Solution | 100 μl |
| Dissolved PEA · AcBz | 20 mg |
| CH₂Cl₂ | 2 ml |
| Surfactant Amount | 5 mg |

This first addition was referred to as the “primary emulsion.” The sample was allowed to stir by shake plate for 5-20 minutes. Once sufficient homogeneity was observed, the primary emulsion was transferred into a canonical vial that contains 0.1% of a surface stabilizer in aqueous media (5-10 mL). These contents are referred to as the “external aqueous phase”. Using a homogenizer at low speed (5000-6000 RPM), the primary emulsion was slowly pipetted into the external aqueous phase, while undergoing low speed homogenization. After 3-5 minutes at 6000 RPM, the total sample (referred to as “the secondary emulsion”) was concentrated in vacuo, to remove the DCM, while encapsulating the PEA.Ac.H nanoparticles within a continuous PEA.Ac.Bz matrix.

Preparation of small Molecules loaded into secondary polymer coatings. In the second route for preparing particles having a triple emulsion structure, the procedure described above for making single emulsion particles was followed for the first step. In the final step, a polymeric coating encapsulating the single emulsion particles (i.e., the water in oil phase) was then prepared.

More particularly, a water in oil phase (primary emulsion) was created. In this case a concentrated mixture of drug (5 mg) and a surfactant (such as DHPC) was prepared first using a minimum volume of water. Then the concentrated mixture was added into a DCM solution of PEA.Ac.Bz, and was subjected to a sonication bath for 5-10 minutes. Once sufficient homogeneity was observed, the contents were added into 5 ml of water while homogenizing. After removal of DCM by vacuum evaporation, a triple emulsion of PEA.Ac.Bz containing aqueous dispersion of drug was obtained.

In another example, a stock sample of PEA.Ac.H nanoparticles with drug was prepared. PEA.Ac.H (25 mg) and drug (5 mg) were dissolved in 2 mL of DCM and mixed with 5 mL of water by sonication for 5-10 minutes. Once sufficient homogeneity was observed, the contents were rotoevaporated to remove DCM. A typical example of preparations made using this method had the following contents.

| | |
|---|---|
| PEA · Ac · H | 25 mg |
| CH₂Cl₂ | 2 mL |
| H₂O | 5 mL |
| Small Molecule Drug | 5 mg |

The above preparation then was subjected to overnight evaporation in a Teflon disk to further reduce the water and yield a volume of approximately 2 mL. An exterior polymer coating, i.e. 25 mg PEA.Ac.Bz in up to 5 mL of DCM, was combined with the primary emulsion and the entire secondary emulsion was stirred by vortexing for no more than 1 minute. Finally, the secondary emulsion was transferred to an aqueous media (10-15 mL) containing 0.1% surface stabilizer, homogenized at 6000 RPM for 5 minutes, and concentrated again in vacuo to remove the second phase of DCM, thus yielding particles having a triple emulsion structure.

### EXAMPLE 5

Drug Capture (50%) by Triple Emulsion The following example illustrates loading of a small molecule drug in a polymer coating. PEA particles containing a high loading of bupivacaine HCl were fabricated by the triple emulsion technique, using a minimal amount of H₂O in the primary emulsion, as compared to the double emulsion protocol (roughly half of the water was used). To stabilize the structure allowing for the reduction in the aqueous phase, the surface stabilizer that aides in solubilizing the drug in the aqueous droplets is dissolved itself in the internal aqueous phase before the drug is added to the internal aqueous phase. In particular, DHPC (amount below) was first dissolved into 100 μL H₂O; then 50 mg of drug was added to the phase. This technique allowed for loading of higher doses of drug in the particles, with even less water than was used in making the same sized double emulsion particles. The following parameters were followed during synthesis:

| | | weight |
|---|---|---|
| Reagent | Mg | equivalence |
| PEA · Ac · Bz | 50 | 50% |
| Bupivacaine | 50 | 50% |
| HCL | | |
| DHPC | 12.4 | 20% of polymer |
| CH₂Cl₂ | 2.5 mL | (2% PEA in |
| (solvent) | | solvent) |
| H₂O | 100 μL | (2:1 drug) |
| DHPC | 16 | 24% of polymer |
| H₂O | | 2/1 ratio to |
| | 5 mL | solvent |

### EXAMPLE 6

Process for Making Triblock Copolymer Micelles with Therapeutic Agents

First, A-B-A type triblock copolymer molecules are formed by conjugating a chain of hydrophobic PEA or PEUR polymer at the center with water soluble polymer chains containing alternating units of PEG and at least one ionizable amino acid, such as lysine or glutamate, at both ends. The triblock copolymer is then purified.

Then micelles are made using the triblock copolymer. The triblock copolymer and at least one bioactive agent, such as a small molecule drug, a protein, peptide, a lipid, a sugar, DNA cDNA or RNA, are dissolved in aqueous solution, preferably in a saline aqueous solution whose pH has been adjusted to a value chosen in such a way that at least a portion of the ionizable amino acids in the water soluble chains is in ionized form to produce a dispersion of the triblock polymer in aqueous solution. Surface stabilizer, such as surfactant or lipid, is added to the dispersion to separate and stabilize particles to be formed. The mixed solution is then stirred with a mechanical or magnetic stirrer, or sonicator. Micelles will be formed in this way, with water-soluble sections mainly on the shell, and hydrophobic sections in the core, maintaining the integrity of micellar particles. The micelles have high porosity for loading of the active agents. Protein and other biologics can be attracted to the charged areas in the water-soluble sections. Micellar particles formed are in the size range from about 20 nm to about 200 nm.

### EXAMPLE 7

Polymer Coating on Particles Made of Different Polymer Mixed with Drug

Use of single emulsion leaves the problem that, although particles can be made very small (from 20 nm to 200 nm), the drug is matrixed in the particles and may elute too quickly. For double and triple emulsion particles, the particles are larger than is prepared by the single emulsion technique due to the aqueous solution inside. However, if the same polymer is used for coating the particles as is used to matrix the drug, the solvent used in making the third emulsion (the polymer coating) will dissolve the matrixed particles, and the coating will become part of the matrix (with drugs in it). To solve this problem, a different polymer than is used to matrix the drug is used to make the coating of the particles and the solvent used in making the polymer coating is selected to be one in which the matrix polymer will not dissolve.

For example, PEA can be dissolved in ethanol but PLA cannot. Therefore, PEA can be used to matrix the drug and PLA can be used as the coating polymer, or vice versa. In another example, ethanol can dissolve PEA but not PEUR and acetone can dissolve PEUR but cannot dissolve PEA. Therefore, PEUR can be used to matrix the drug and PEA can be used as the coating polymer, or vice versa.

Therefore, the general process to be used is as follows. Using polymer A, prepare particles in solution (aqueous if polymer A is PEA or PEUR) using a single emulsion procedure to matrix drug or other bioactive agent in the polymer particles. Dry out the solvent by lyophilization to obtain dry particles. Disperse the dry particles into a solution of polymer B in a solvent that does not dissolve the polymer A particles. Emulsify the mixture in aqueous solution. The resulting particles will be nanoparticles with a coating of polymer B on particles of polymer A, which contain matrixed drug.

### EXAMPLE 8

In this example a PEA polymer containing a residue of β-Estradiol in the main PEA polymer backbone was prepared.

Materials 17-β-estradiol (estra-1,3,5(10)-triene-3,17β-diol), L-lysine, benzyl alcohol, sebacoyl chloride, 1,6-Hexanediol, p-nitrophenol, triethylamine, 4-N,N-(dimethylamino)pyridine (DMAP), N,N′-dicyclohexylcarbodiimide (DCC), anhydrous N,N-dimethylformamide (DMF), anhydrous dichloromethane (DCM), trifluoroacetic acid (TFA), p-toluenesulfonic acid monohydrate (Aldrich Chemical Co., Milwaukee, WI), anhydrous toluene, Boc-L-leucine monohydrate (Calbiochem-Novabiochem, San Diego, Calif) were used without further purification. Other solvents, ether and ethyl acetate (Fisher Chemical, Pittsburgh, Pa.).

Synthesis of Monomers and Polymers Synthesis of bioactive PEAs involved three basic steps: (1) synthesis of bis-electrophiles: di(p-nitrophenyl) esters of dicarboxylic acid (here of sebacic acid, compound 1); (2) synthesis of bis-nucleophiles: di-p-toluenesulfonic acid salts (or di-TFA salt) of bis(L-leucine)-diol-diesters (compounds 3 and 5) and of L-lysine benzyl ester (compound 2); and (3) solution polycondensation of the monomers obtained in steps (1) and (2).

Synthesis of di-p-nitrophenyl esters of sebacic acid (compound 1) Di-p-nitrophenyl ester of sebacic acid was prepared by reaction of sebacoyl chloride with p- nitrophenol as described previously (Katsarava et al. J. Polym. Sci. Part A: Polym. Chem. (1999) 37. 391-407) (scheme 4): 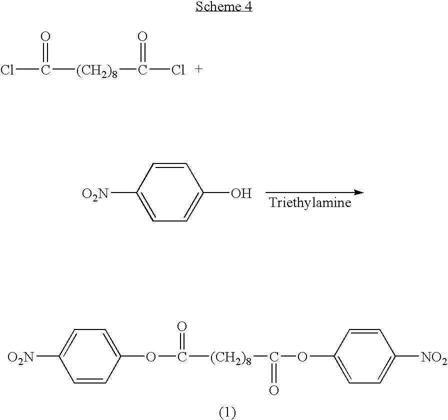
A di-p-toluenesulfonic acid salt of L-lysine benzyl ester was prepared as described earlier (U.S. Pat. No. 6,503,538) by refluxing of benzyl alcohol, toluenesulfonic acid monohydrate and L-lysine monohydrochloride in toluene, while applying azeotropic removal of generated water (scheme Scheme 5).

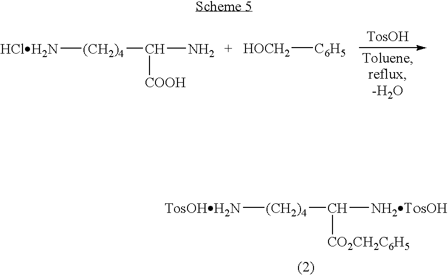

Synthesis of acid salts of bis(α-amino acid) diesters (3), (5) Di-p-toluenesulfonic acid salt of bis(L-leucine) hexane-1,6-diester (compound 3) was prepared by modified procedure of the previously published method as shown in scheme 3.

L-Leucine (0.132 mol), p-toluenesulfonic acid monohydrate (0.132 mol) and 1,6-hexanediol (0.06 mol) in 250 mL of toluene were placed in a flask equipped with a Dean-Stark apparatus and overhead stirrer. The heterogeneous reaction mixture was heated to reflux for about 12 h until 4.3 mL (0.24 mol) of water evolved. The reaction mixture was then cooled to room temperature, filtered, washed with acetone, and recrystallized twice from methanol/toluene 2:1 mixture. Yields and Mp were identical to published data (Katsarava et al., supra) (see scheme 6). 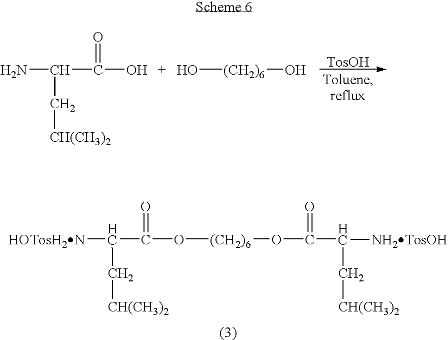
A di-TFA salt of bis-(L-leucine)- estradiol-3,17β-diester (compound 5) was prepared by a two step reaction. 17β-Estradiol was first reacted with boc-protected L-leucine, applying carbodiimide mediated esterification, to form compound 4. In a second step, boc groups were deprotected using TFA, converting at the same time into a di-TFA salt of di-amino monomer (compound 5) (see scheme 7).

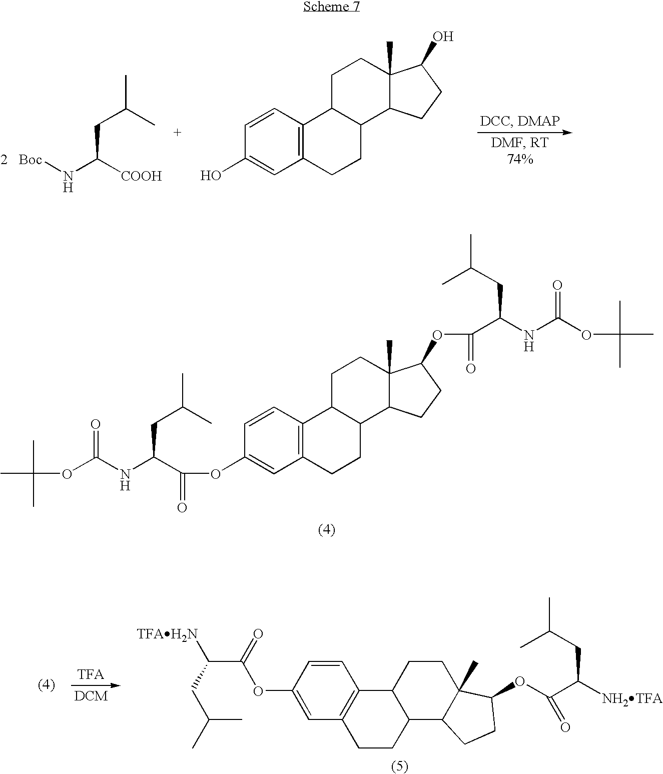

Preparation of Bis(Boc-L-leucine)estradiol-3,17β-diester (4) 1.5 g (5.51 mmol) of 17β-estradiol, 3.43 g (13.77 mmol) Boc-L-leucine monohydrate and 0.055 g (0.28 mmol) of p-toluenesulfonic acid monohydrate were dissolved into 20 mL of dry N,N-dimethylformamide at room temperature under a dry nitrogen atmosphere. To this solution 10 g of molecular sieves were added and stirring continued for 24 h. Then, 0.067 g of DMAP and 5.4 g of (26.17 mmol) DCC were introduced into the reaction solution and stirring was continued. After 6 h (no discoloration of the reaction was observed), 1 mL of acetic acid was added to destroy the excess of DCC. Precipitated urea and sieves then were filtered off and filtrate poured in 80 mL of water. Product was extracted three times with 30 mL of ethylacetate, dried over sodium sulfate, solvent evaporated, and the product was subjected to chromatography on a column (7:3 hexanes: ethylacetate). A colorless glassy solid of pure compound 4 obtained in a 2.85 g, 74% yield and 100% purity (TLC) and was further converted to compound 5.

DI-TFA salt of bis(L-leucine)estradiol-3,17β-diester (compound 5). Deprotection of Boc-protected monomer (compound 4) was carried out substantially quantitatively in 10 mL of dry dichloromethane, by adding 4 mL of dry TFA. After 2 h of stirring at room temperature, a homogenous solution was diluted with 300 mL of anhydrous ether and left in a cold room over night. Precipitated white crystals were collected, washed twice with ether, and dried in a vacuum oven at 45° C. Yield 2.67 g (90%). Mp=187.5° C.

Polymer Synthesis. Synthesis of therapeutic PEA was carried out in DMF in mild conditions (60° C.): 4 eq. activated di-acid monomer (compound 1) was reacted with combinations of the di-amino monomers 1.5 eq. (compound 2), 1.5 eq. (compound 5) and 1 eq. of (compound 3).

Triethylamine 1.46 mL (10.47 mmol) was added at once to the mixture of monomers (compound 1) (4.986 mmol), (compound 2) (1.246 mmol), (compound 3) (1.869 mmol), (compound 5) (1.869 mmol) in 3 mL of dry DMF and the solution was heated to 60° C. while stirring. The reaction vial was kept at the same temperature for 16 h. A yellow viscous solution was formed then was cooled down to room temperature, diluted with 9 mL of dry DMF, added 0.2 mL of acetic anhydride, and after 3 h precipitated out three times: first in water, then from ethanol solution into ethylacetate and lastly, from chloroform in ethyl acetate. A colorless hydrophobic polymer was cast as a film from chloroform: ethanol (1:1) mixture and dried in vacuum. Yield: 1.74 g (70%).

Materials Characterization The chemical structure of monomers and polymer were characterized by standard chemical methods. NMR spectra were recorded by a Bruker AMX-500 spectrometer (Numega R. Labs Inc. San Diego, Calif.) operating at 500 MHz for ¹H NMR spectroscopy. Deuterated solvents CDCl₃ or DMSO-d₆ (Cambridge Isotope Laboratories, Inc., Andover, Mass.) were used with tetramethylsilane (TMS) as internal standard.

Melting points of synthesized monomers were determined on an automatic Mettler-Toledo FP62 Melting Point Apparatus (Columbus, Ohio). Thermal properties of synthesized monomers and polymers were characterized on Mettler-Toledo DSC 822e differential scanning calorimeter. Samples were placed in aluminum pans. Measurements were carried out at a scanning rate of 10° C./min under nitrogen flow.

The number and weight average molecular weights (Mw and Mn) and molecular weight distribution of synthesized polymer was determined by Model 515 gel permeation chromatography (Waters Associates Inc. Milford, Mass.) equipped with a high pressure liquid chromatographic pump, a Waters 2414 refractory index detector. 0.1% of LiCl solution in N,N-dimethylacetamide (DMAc) was used as eluent (1.0 mL/min). Two Styragel® HR 5E DMF type columns (Waters) were connected and calibrated with polystyrene standards.

Tensile Properties: tensile strength, elongation at break and Young's Modulus were measured on a tensile strength instrument (Chatillon TCD200, integrated with a PC (Nexygen™ FM software)(Chatillon, Largo, Fla.) at a crosshead speed of 100 mm/min. The load capacity was 50 lbs. The film (4×1.6 cm) had a dumbbell shape and thickness of about. 0.125 mm.

Results: Four different monomers were copolymerized by polycondensation of activated monomers, affording copoly PEA containing 17% w/w steroid-diol load on a total polymer weight basis. Chemical structure of the product therapeutic polymer composition, containing fragments of 17β-estradiol, L-Leucine, L-Lysine (OBn), 1,6-hexanediol and sebacic acid is depicted in Formula (XIX). 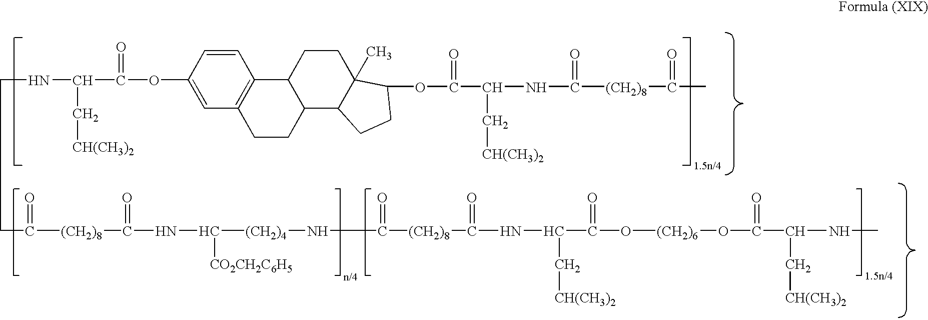
Three monomers: bis-p-toluenesulfonic acid salts of L-lysine-benzyl ester (compound 2), bis(L-leucine) 1,6-hexane diester (compound 3), and bis(p-nitrophenyl) sebacate (compound 1) were prepared according to the literature and characterized by melting point and proton NMR spectroscopy. Results were in agreement with those reported in literature.

In this example a PEA polymer containing a residue of 17β-Estradiol in the main polymer backbone was prepared, where both hydroxyls of the diol steroid were incorporated into monomer via ester bonds using a carbodiimide technique. The final monomer introduced into the polymerization reaction was a TFA salt. After polycondensation, a high molecular weight copolymer was obtained. Gel permeation chromatography yielded an estimated (PS) weight average Mw=82,000 and polydispersity PDI=1.54. The product copolymer was partially soluble in ethanol (when dry), well soluble in chloroform, chloroform: ethanol 1:1 mixture, dichloromethane, and in polar aprotic organic solvents: DMF, DMSO, DMAc.

Glass transition temperature was detected at Tg=41° (midpoint, taken from the second heating curve) and a sharp melting endotherm was detected at 220° C. by Differential scanning calorimetry (DSC) analysis. This result leads to the conclusion that the polymer has semi-crystalline properties.

The therapeutic polymer formed a tough film when cast from chloroform solution. Tensile characterization yielded the following results: Stress at break 28.1 MPa, Elongation 173%, Young's Modulus 715 MPa.

### EXAMPLE 9

This Example illustrates synthesis of a therapeutic PEUR polymer composition (Formula V) containing a therapeutic diol in the polymer backbone is illustrated in this example. A first monomer used in the synthesis is a di-carbonate of a therapeutic diol with a general chemical structure illustrated by formula 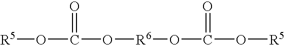
is formed using a known procedure (compound (X) as described in U.S. Pat. No. 6,503,538) wherein R⁵ is independently (C₆-C₁₀) aryl (e.g. p-nitrophenol, in this example), optionally substituted with one or more nitro, cyano, halo, trifluoromethyl or trifluoromethoxy; and at least some of p-nitrophenol. At least some of R⁶ is a residue of a therapeutic diol as described herein, depending upon the desired drug load. In the case where all of R⁶ is not the residue of a therapeutic diol, each diol would first be prepared and purified as a separate monomer. For example, di-p-nitrophenyl-3,17β-estradiol-dicarbonate (compound 6) can be prepared by the method of Scheme 8 below: 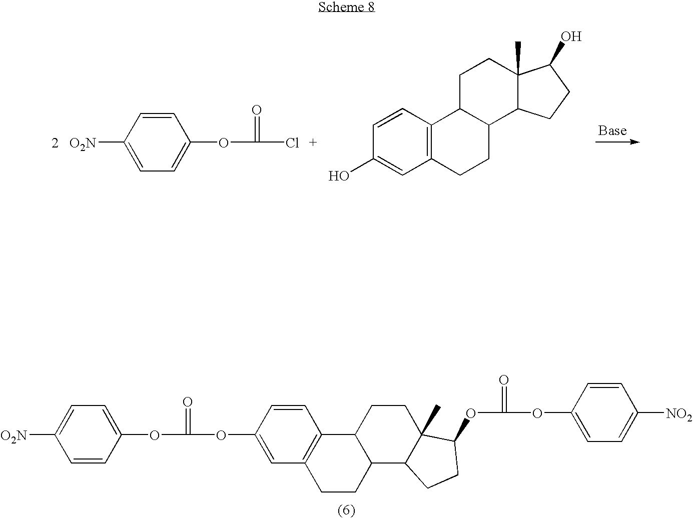
Polycondensation of compound X from U.S. Pat. No. 6,503,538 (in our example compound 6) with the monomers described above yields an estradiol-based co-poly(ester urethane) PEUR (compound 1 1): 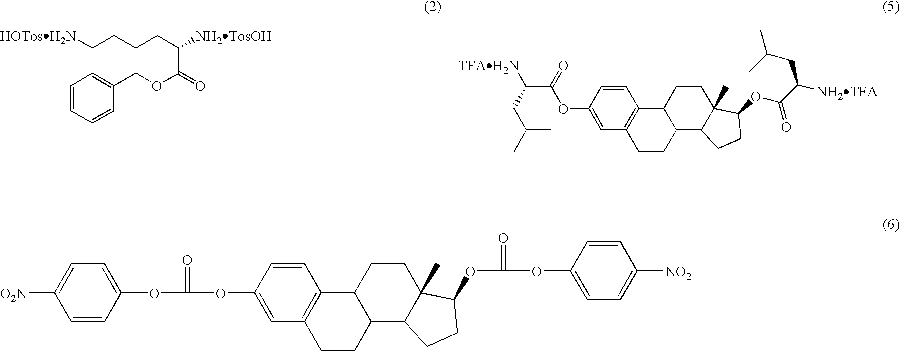
wherein the reaction scheme is as follows

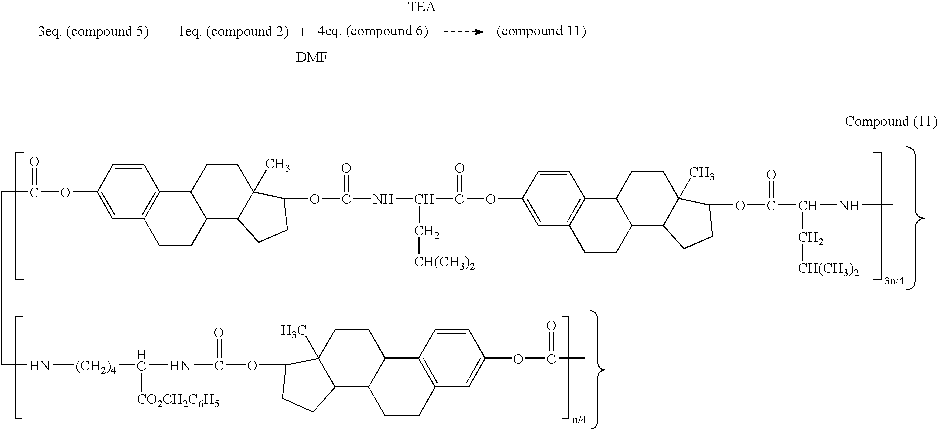

### EXAMPLE 10

Monomer Synthesis for Preparation of PEU Polymers

Preparation of diamine type monomers--di-p-toluenesulfonic acid salt of L-lysine benzyl ester (L-Lys(OBn), Compound 2) and di-toluenesulfonic acid salt of bis(L-leucine)-hexane-1,6-diester, (compound 3)—were described in previous Example 8.

Preparation of Di-p-toluenesulfonic acid salt of bis(L-leucine)-1,4:3,6-dianhydrosorbitol-diester (Compound 7) was conducted as described previously (Z. Gomurashvili et al. J. Macromol. Sci.—Pure. Appl. Chem. (2000) A37: 215-227). 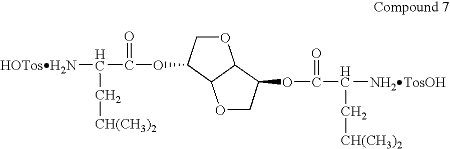
wherein L-leucine (0.132 mol), p-toluenesulfonic acid monohydrate (0.135 mol) and isosorbide (0.06 mol) in 250 mL of toluene were placed in a flask equipped with a Dean-Stark apparatus and overhead stirrer. The heterogeneous reaction mixture was heated to reflux for about. 12 h until 4.3 mL (0.24 mol) of water evolved. The reaction mixture was then cooled to room temperature, filtered, washed with acetone and recrystallized twice from methanol/toluene 2:1. mix. Yields and Mp were identical to published data (Z. Gomurashvili et al. supra).

### EXAMPLE 11

Preparation of PEU 1-L-Leu-6 (Polymer Entry #2, Table 2)

To a suspension of 6.89 g (10 mmol) of di-p-toluenesulfonic acid salt of bis(L-leucine)-□-hexanediol-diester in 150 mL of water, 4, 24 g (40 mmol) of anhydrous sodium carbonate was added, stirred at room temperature for 30 min. and cooled to 2° C. to 0° C. In parallel, a solution of 0.9893 g (10 mmol) of phosgene in 35 mL of chloroform was cooled to 10 to 15° C. The first solution was placed into a reactor for interfacial polycondensation and the second solution was quickly added in bolus and stirred briskly for 15 min. Then the chloroform layer was separated, dried, over anhydrous Na₂SO₄, and filtered. The obtained solution was evaporated and the polymer yield was dried in vacuum at 45° C. Yield was 82%. For ¹H and ¹³C NMR see FIG. 2 and FIG. 3. Elemental analysis: for C₁₉H₃₄N₂O₅, calculated values: C: 61.60%, H: 9.25%, N: 7.56%; Found values: C: 61.63%, H: 8.90%, N: 7.60.

### EXAMPLE 12

Preparation of PEU 1-L-Leu-DAS (Polymer: Entry #5, Table 2)

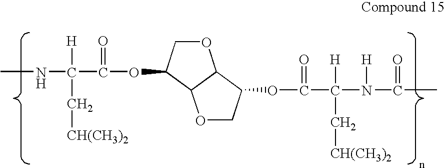

A cooled solution (ice-bath) of 5 g (6.975 mmole) of bis (L-leucine)-1,4:3,6-dianhydrosorbitol-diester (compound 7) and 2.4 g of sodium carbonate in 40 mL of water was prepared. To the cooled solution, 70 mL of chloroform was added with vigorous stirring and then 3.7 mL of 20% phosgene solution in toluene (Fluka) was introduced. Poly(ester urea) formed rapidly with evolution of heat. After the reaction had been stirred for 10 min, the organic layer was rotoevaporated and residual polymer was filtered, washed several times with water, and dried in vacuum over night. Yield of product was 1.6 g. (57%). Polymer properties are as summarized in Table 2.

### EXAMPLE 13

This example describes a degradation study conducted to compare degradation rates over time of a PEU polymer 1-L-Leu-4. Circular PEU films of 4 cm diameter and 400-500 mg each, were placed into the glass beakers containing 10 ml of 0.2 M phosphate buffer solution of pH 7.4 with 4 mg of an enzyme, either α-chymotrypsin or lipase, or without enzymes. The glass vessels were maintained at 37° C. Films were removed from the enzyme solution after predetermined time, dried up to constant weights, and weighed. Then the films were placed into the fresh solution of either enzyme or pure buffer and all the procedures described above were repeated. Weight changes per unit surface area of the sample were calculated and represented graphically vs. biodegradation time. The results of the study showed that the PEU polymer has a degradation profile that is almost zero order, corresponding to a surface degradation profile.

All publications, patents, and patent documents are incorporated by reference herein, as though individually incorporated by reference. The invention has been described with reference to various specific and preferred embodiments and techniques. However, it should be understood that many variations and modifications might be made while remaining within the spirit and scope of the invention.

Although the invention has been described with reference to the above examples, it will be understood that modifications and variations are encompassed within the spirit and scope of the invention. Accordingly, the invention is limited only by the following claims.

What is claimed is:

1. An intraocular polymer delivery composition comprising at least one ophthalmologic agent dispersed in at least one biodegradable polymer, wherein the polymer comprises at least one of a poly(ester amide) (PEA) having a chemical formula described by structural formula (I), 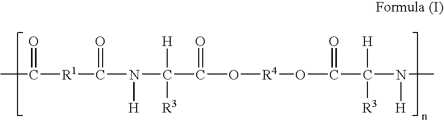
wherein n ranges from about 5 to about 150; R¹ is independently selected from residues of α,ω-bis (4-carboxyphenoxy) (C₁-C₈) alkane, 3,3′-(alkanedioyidioxy) dicinnamic acid or 4,4′-(alkanedioyldioxy) dicinnamic acid, residues of α,ω-alkylene dicarboxylates of formula (III), (C₂-C₂₀) alkylene, (C₂-C₂₀) alkenylene or a saturated or unsaturated residues of therapeutic di-acids and combinations thereof; wherein R⁵ and R⁶ in Formula (III) are independently selected from (C₂-C₁₂) alkylene or (C₂-C₁₂) alkenylene; the R³s in individual n monomers are independently selected from the group consisting of hydrogen, (C₁-C₆) alkyl, (C₂-C₆) alkenyl, (C₆-C₁₀) aryl (C₁-C₆) alkyl and —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), saturated or unsaturated therapeutic di-acid residues and combinations thereof; 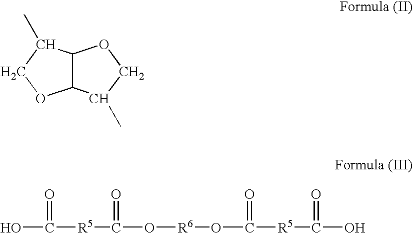
or a PEA having a chemical formula described by structural formula (IV): 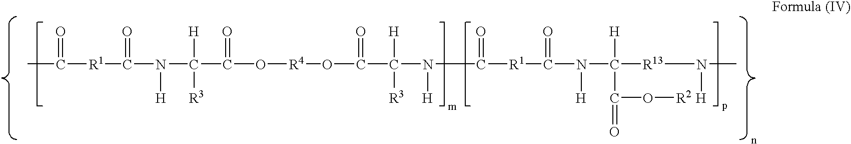
wherein n ranges from about 5 to about 150, m ranges about 0.1 to 0.9: p ranges from about 0.9 to 0.1; wherein R¹ is independently selected from residues of α,ω-bis (4-carboxyphenoxy) (C₁-C₈) alkane, 3,3′-(alkanedioyidioxy) dicinnamic acid or 4,4′-(alkanedioyidioxy) dicinnamic acid, residues of α,ω-alkylene dicarboxylates of formula (III), (C₂-C₂₀) alkylene, (C₂-C₂₀) alkenylene or a saturated or unsaturated residues of therapeutic di-acids and combinations thereof; wherein R⁵ and R⁶ in Formula (III) are independently selected from (C₂-C₁₂) alkylene or (C₂-C₁₂) alkenylene; each R² is independently hydrogen, (C₁-C₁₂) alkyl, (C₂-C₈) alkyloxy (C₂-C₂₀) alkyl, (C₆-C₁₀) aryl or a protecting group; the R³s in individual m monomers are independently selected from the group consisting of hydrogen, (C₁-C₆) alkyl, (C₂-C₆) alkenyl, (C₆-C₁₀) aryl (C₁-C₆) alkyl and -(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated or unsaturated therapeutic diols and combinations thereof; and R¹³ is independently (C₁-C₂₀) alkyl or (C₂-C₂₀) alkenyl, for example, (C₃-C₆) alkyl or (C₃-C₆) alkenyl;
or a poly(ester urethane) (PEUR) having a chemical formula described by structural formula (V), 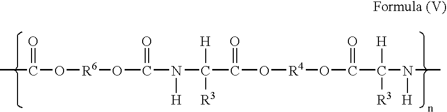
and wherein n ranges from about 5 to about 150; wherein the R³s within an individual n monomer are independently selected from the group consisting of hydrogen, (C₁-C₆) alkyl, (C₂-C₆)alkenyl, (C₆-C₁₀) aryl(C₁-C₆) alkyl and —(CH₂)₂S(CH₃); R⁴ and R⁶ is selected from the group consisting of (C₂-C₂₀) alkylene, (C₂-C₂₀) alkenylene or (C₂-C₈) alkyloxy (C₂-C₂₀) alkylene, and bicyclic-fragments of 1,4:3,6-dianhydrohexitols of structural formula (II) a residue of a saturated or unsaturated therapeutic diol, and mixtures thereof;
or a PEUR having a chemical structure described by general structural formula (VI), 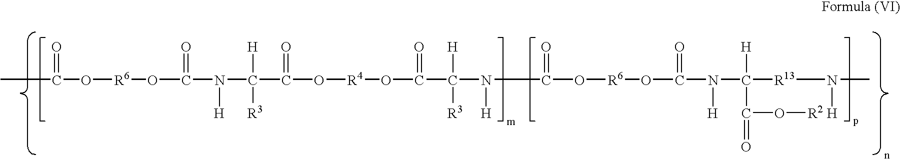
wherein n ranges from about 5 to about 150, m ranges about 0.1 to about 0.9: p ranges from about 0.9 to about 0. 1; R² is independently hydrogen, (C₁-C₁₂) alkyl, (C₂-C₈) alkyloxy (C₂-C₂₀) alkyl, (C₆-C₁₀) aryl or a protecting group; the R³s within an individual m monomer are independently selected from the group consisting of hydrogen, (C₁-C₆) alkyl, (C₂-C₆) alkenyl, (C₆-C₁₀) aryl (C₁-C₆) alkyl and —(CH₂)₂S(CH₃); R⁴ and R⁶ is independently selected from (C₂-C₂₀) alkylene, (C₂-C₂₀) alkenylene or (C₂-C₈) alkyloxy (C₂-C₂₀) alkylene, bicyclic-fragments of 1,4:3,6-dianhydrohexitols of structural formula (II), a residue of a saturated or unsaturated therapeutic diol, and mixtures thereof; and R¹³ is independently (C₁-C₂₀) alkyl or (C₂-C₂₀) alkenyl, for example, (C₃-C₆) alkyl or (C₃-C₆) alkenyl;
or a poly(ester urea) (PEU) having a chemical formula described by structural formula 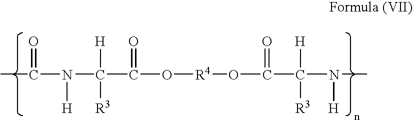
wherein n is about 10 to about 150; the R³s within an individual n monomer are independently selected from hydrogen, (C₁-C₆) alkyl, (C₂-C₆) alkenyl, (C₆-C₁₀) aryl (C₁-C₆)alkyl and —(CH₂)₂S(CH₃); R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II) and mixtures of thereof;
or a PEU having a chemical formula described by structural formula (VIII), 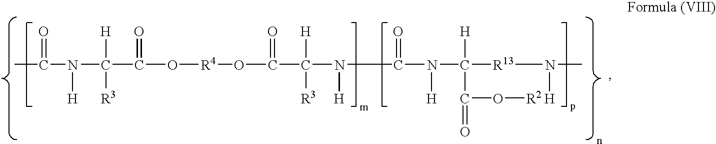
wherein m is about 0.1 to about 1.0; p is about 0.9 to about 0.1; n is about 10 to about 150; each R² is independently hydrogen, (C₁-C₁₂) alkyl, (C₂-C₈) alkyloxy (C₂-C₂₀) alkyl, (C₆-C₁₀) aryl or a protecting group; and the R³s within an individual m monomer are independently selected from hydrogen, (C₁-C₆) alkyl, (C₂-C₆) alkenyl, (C₆-C₁₀) aryl (C₁-C₆)alkyl and —(CH₂)₂S(CH₃); R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II), or a mixture thereof; and R¹³ is independently (C₁-C₂₀) alkyl or (C₂-C₂₀) alkenyl, for example, (C₃-C₆) alkyl or (C₃-C₆) alkenyl;

2. The composition of claim 1, wherein the composition is formulated for intraocular administration in the form of a liquid dispersion.

3. The composition of claim 2, wherein the liquid dispersion is a dispersion of polymer particles.

4. The composition of claim 3, wherein in the particles have an average diameter in the range from about 10 nanometers to about 1000 microns.

5. The composition of claim 3, further comprising a covering water soluble molecule conjugated to the polymer on the exterior of the particles.

6. The composition of claim 3, wherein a particle includes from about 5 to about 150 molecules of the ophthalmologic agent per polymer molecule chain.

7 The composition of claim 3, wherein the composition is formulated as lyophilized polymer particles.

8. The composition of claim 3 wherein the composition is formulated as micro- or nano-particles.

9. The composition of claim 1, wherein the composition forms a time release polymer depot when injected intraocularly.

10. The composition of claim 1, wherein a residue of the ophthalmologic agent is the therapeutic diol incorporated into the backbone of the polymer.

11. The composition of claim 1, wherein the polymer has the chemical formula described by structural formula (I), (V) or (VII) and R³s in at least one monomer n is CH₂Ph.

12. The composition of claim 1, wherein the 1,4:3,6-dianhydrohexitol of structural formula (II) is derived from D-glucitol, D-mannitol, or L-iditol.

13. The composition of claim 1, wherein the composition biodegrades over a period of about twenty-four hours to about three years.

14. The composition of claim 1, wherein a polymer molecule in the particles has an average molecular weight in range from about 5,000 to about 300,000.

15. The composition of claim 1, wherein the ophthalmologic agent is conjugated to at least one of the polymers.

16. The composition of claim 1, further comprising at least one bioactive agent dispersed in the polymer(s).

17. The composition of claim 1, wherein the composition further comprises a pharmaceutically acceptable vehicle.

18. The composition of claim 1, wherein the composition forms a solid.

19. The composition of claim 18, wherein the composition has a thickness of about 0.1 to about 2.5 mm.

20. The composition of claim 18, further comprising at least one coating layer of a biodegradable, biocompatible polymer.

21. The composition of claim 20, wherein the polymer of the coating layer has a chemical formula described by structural formulas (I) and (IV-VIII).

22. The composition of claim 20, wherein the coating layer is free of bioactive agents.

23. The composition of claim 20, wherein the polymer of the coating layer and the carrier layer are the same.

24. The composition of claim 20, further comprising at least one barrier layer of a polymer that is insoluble in solvent(s) for the polymer(s) of the carrier layer and the coating layer, but dissolves in intraocular conditions, wherein the barrier layer is between the carrier layer and the coating layer.

25. The composition of claim 24, wherein there are multiple sets of the coating layer and barrier layer, with the coating layer being exterior in each successive set.

26. The composition of claim 25, wherein successive outwardly lying layers of the multiple sets of layers encompass all interior layers.

27. The composition of claim 25, wherein the shape of the composition is substantially rectangular or cylindrical with a smallest dimension of about 1 mm.

28. The composition of claim 21, wherein the composition is sized for injection via a pharmaceutical syringe needle having a bore of about 18 to 25 gauge.

29. The composition of claim 21, wherein the composition is fabricated in the shape of a disc, sheet, film, fiber or tube.

30. A method of delivering at least one ophthalmologic agent to the interior or exterior of the eye of a subject, said method comprising:
administering the composition of any one of claims 1-34 to the interior or exterior of the eye of the subject for controlled release of the ophthalmologic agent therein.

31. The method of claim 30, wherein the composition is administered subconjunctivally.

32. The method of claim 30, wherein the composition is administered via a pharmaceutical syringe needle.

33. The method of claim 30, wherein the composition is implanted for subtenon delivery.

34. The method of claim 30, wherein the composition is applied topically.
